(19) United States
(12) Patent Application Publication
(10) Pub. No.: US 2006/0142393 A1
(43) Pub. Date: Jun. 29, 2006

(54) Bis(thio-hydrazide amides) for treatment of hyperplasia

(76) Inventors: Matthew L. Sherman, Newton, MA (US); Farid Vaghefi, Burlington, MA (US); Lan Bo Chen, Lexington, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 11/226,929

(22) Filed: Sep. 14, 2005

Related U.S. Application Data
(60) Provisional application No. 60/610,270, filed on Sep. 16, 2004.

(51) Int. Cl. A61K 31/16

(52) U.S. Cl. 514/599

(57) ABSTRACT

Methods and medical devices for treating a proliferative disorder in a subject, e.g., restenosis in a blood vessel that has been implanted with a stent, employ a bis(thio-hydrazide amide) represented by Structural Formula I or a pharmaceutically acceptable salt or solvate thereof. 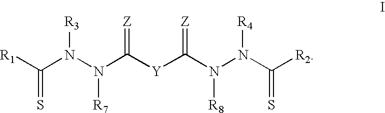 Y is a covalent bond or an optionally substituted straight chained hydrocarbyl group, or, Y, taken together with both >C=Z groups to which it is bonded, is an optionally substituted aromatic group.

R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring.

R₇-R₈ are independently —H, an optionally substituted aliphatic group, or an optionally substituted aryl group. Z is O or S.

### RELATED APPLICATION

This application claims the benefit of U.S. Provisional Application No. 60/610,270, filed on Sep. 16, 2004. The entire teachings of the above application are incorporated herein by reference.

### BACKGROUND OF THE INVENTION

Numerous non-cancer diseases involve excessive or hyperproliferative cell growth, termed hyperplasia. Examples include hyperproliferative skin disorders such as psoriasis and its varied clinical forms, Reiter's syndrome, pityriasis rubra pilaris, and hyperproliferative variants of disorders of keratinization (e.g., actinic keratosis, senile keratosis), and the like. Other examples include reproductive system-associated disorders such as benign prostatic hyperplasia, ovarian cysts, and the like. Moreover, proliferative smooth muscle disorders, such as intimal smooth muscle cell hyperplasia, can lead to blockage in, for example, the urethra, the bile duct, and blood vessels, particularly following biologically- or mechanically-mediated tissue injury.

One common type of non-cancerous proliferative disorder is restenosis, such as that associated with balloon angioplasty. In subjects with obstructive coronary artery disease, abatement of the chest pain associated with blocked blood vessels can sometimes be achieved by insertion of a stent-equipped angioplasty balloon. Inflating the balloon opens the blood vessel and installs the stent to keep the blood vessel open after removal of the balloon. The benefit is often temporary, however, because stented blood vessels can become reblocked due to cell growth in response to tissue injury from the insertion. This process is termed restenosis.

In some cases, hyperproliferative cell growth can be inhibited by radiation therapy, but such therapy has practical and cost limitations, as well as questionable long term safety. Certain drugs can sometimes inhibit hyperproliferative cell growth. For example, particular drug-coated stents are commercially available to avoid restenosis. However, current drugs have limited effectiveness, and restenosis can still occur with marketed drug-coated stents. It is desireable to have improved treatments that inhibit hyperproliferative cell growth.

### SUMMARY OF THE INVENTION

Disclosed are methods and medical devices that employ bis(thio-hydrazide amides) for treating proliferative disorders. The bis(thio-hydrazide amides) are represented by Structural Formula I:

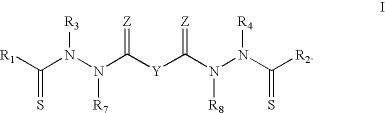

Y is a covalent bond or an optionally substituted straight chained hydrocarbyl group, or, Y, taken together with both >C=Z groups to which it is bonded, is an optionally substituted aromatic group.

R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring.

R₇-R₈ are independently —H, an optionally substituted aliphatic group, or an optionally substituted aryl group.

Z is O or S.

As used herein, the term “bis(thio-hydrazide amide)” also includes pharmaceutically acceptable salts and solvates of the compounds represented by Structural Formula I.

One embodiment of the invention is a method of treating a non-cancerous proliferative disorder in a subject comprising administering to the subject an effective amount of the bis(thio-hydrazide amide). In one example, the disorder is a proliferative vascular disorder, e.g., restenosis in a blood vessel that has been treated with balloon angioplasty.

Another embodiment of the invention is a medical device that comprises a reservoir, a coating composition or a controlled release polymer matrix that comprises the bis(thio-hydrazide amide), and can release the bis(thio-hydrazide amide) in vivo. In some embodiments, the medical device is a stent.

Another embodiment of the invention is a method of treating a proliferative cell disorder (e.g., a cancerous or non-cancerous disorder) at a treatment site in a subject comprising contacting the subject with the medical device and releasing the compound in vivo.

The disclosed inventions have many advantages. The methods and devices described herein are believed to be effective for treating non-cancerous proliferative disorders. In addition, the medical device can deliver the bis(thio-hydrazide amide) directly to a treatment site, for example, a blood vessel at an angioplasty treatment site that is at risk of restenosis. In this manner, a high local concentration of the bis(thio-hydrazide amide) can be achieved at the treatment site while minimizing global drug concentration in the body. It is believed that therapeutic effectiveness can thereby be increased and side effects can be minimized. Other advantages will become clear from the detailed description that follows.

### BRIEF DESCRIPTION OF THE DRAWINGS

FIG. 1 is a graph showing the inhibitory effect of Compound (16) (♦) compared to vehicle (●) on average tumor size in nude mice (CD-1 nu/nu) over time. The tumor volume is in mm³ and the time is in days after having begun treatment. The tumors are from the multi-drug resistant human uterine sarcoma MES—SA/DX5.

FIG. 2 is a graph showing the inhibitory effect of Compound (1) (50 mg/kg) compared to vehicle (●) ; Epothilone D (5 mg/kg) (♦); and Compound (1) and Epothilone(5 mg/kg) (∘) on the average tumor volume in milliliters over time (in days) in nude mice (CD-1 nu/nu). The tumors were generated from the human breast tumor cell line MDA-435.

FIG. 3 is a graph showing the average percent weight change in nude mice (CD-1 nu/nu) over time after having been treated with vehicle (●); Epothilone D (5 mg/kg) (♦); and Compound (1) (50 mg/kg) and Epothilone(5 mg/kg) (>>). The mice were being treated for tumors generated tumors generated from the human breast tumor cell line MDA-435.

### DETAILED DESCRIPTION OF THE INVENTION

The bis(thio-hydrazide amides) employed in the disclosed invention are represented by Structural Formula I.

A “straight chained hydrocarbyl group” is an alkylene group, i.e., —(CH₂)_{y}—, with one, or more (preferably one) internal methylene groups optionally replaced with a linkage group. y is a positive integer (e.g., between 1 and 10), preferably between 1 and 6 and more preferably 1 or 2. A “linkage group” refers to a functional group which replaces a methylene in a straight chained hydrocarbyl. Examples of suitable linkage groups include a ketone (—C(O)—), alkene, alkyne, phenylene, ether (—O—), thioether (—S—), or amine (—N(R^{a})—), wherein R^{a} is defined below. A preferred linkage group is —C(R₅R₆)—, wherein R₅ and R₆ are defined above. Suitable substitutents for an alkylene group and a hydrocarbyl group are those which do not substantially interfere with the anti-cancer activity of the disclosed compounds. R₅ and R₆ are preferred substituents for an alkylene or hydrocarbyl group represented by Y.

An aliphatic group is a straight chained, branched or cyclic non-aromatic hydrocarbon which is completely saturated or which contains one or more units of unsaturation. Typically, a straight chained or branched aliphatic group has from 1 to about 20 carbon atoms, preferably from 1 to about 10, and a cyclic aliphatic group has from 3 to about 10 carbon atoms, preferably from 3 to about 8. An aliphatic group is preferably a straight chained or branched alkyl group, e.g, methyl, ethyl, n-propyl, iso-propyl, n-butyl, sec-butyl, tert-butyl, pentyl, hexyl, pentyl or octyl, or a cycloalkyl group with 3 to about 8 carbon atoms. A C1-C20 straight chained or branched alkyl group or a C3-C8 cyclic alkyl group is also referred to as a “lower alkyl” group.

The term “aromatic group” may be used interchangeably with “aryl,” “aryl ring,” “aromatic ring,” “aryl group” and “aromatic group.” Aromatic groups include carbocyclic aromatic groups such as phenyl, naphthyl, and anthracyl, and heteroaryl groups such as imidazolyl, thienyl, furanyl, pyridyl, pyrimidy, pyranyl, pyrazolyl, pyrroyl, pyrazinyl, thiazole, oxazolyl, and tetrazole. The term “heteroaryl group” may be used interchangeably with “heteroaryl,” “heteroaryl ring,” “heteroaromatic ring” and “heteroaromatic group.” The term “heteroaryl,” as used herein, means a mono-or multi-cyclic aromatic heterocycle which comprise at least one heteroatom such as nitrogen, sulfur and oxygen, but may include 1, 2, 3 or 4 heteroatoms per ring. Aromatic groups also include fused polycyclic aromatic ring systems in which a carbocyclic aromatic ring or heteroaryl ring is fused to one or more other heteroaryl rings. Examples include benzothienyl, benzofuranyl, indolyl, quinolinyl, benzothiazole, benzooxazole, benzimidazole, quinolinyl, isoquinolinyl and isoindolyl.

The term “arylene” refers to an aryl group which is connected to the remainder of the molecule by two other bonds. By way of example, the structure of a 1,4-phenylene group is shown below:

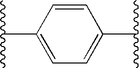

Substituents for an arylene group are as described below for an aryl group.

Non-aromatic heterocyclic rings are non-aromatic rings which include one or more heteroatoms such as nitrogen, oxygen or sulfur in the ring. The ring can be five, six, seven or eight-membered. Examples include tetrahydrofuranyl, tetrahyrothiophenyl, morpholino, thiomorpholino, pyrrolidinyl, piperazinyl, piperidinyl, and thiazolidinyl.

Suitable substituents on an aliphatic group (including an alkylene group), non-aromatic heterocyclic group, benzylic or aryl group (carbocyclic and heteroaryl) are those which do not substantially interfere with the anti-cancer activity of the disclosed compounds. A substituent substantially interferes with anti-cancer activity when the anti-cancer activity is reduced by more than about 50% in a compound with the substituent compared with a compound without the substituent. Examples of suitable substituents include —R^{a}, —OH, —Br, —Cl, —I, —F, —OR^{a}, —O—COR^{a}, —COR^{a}, —CN, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NR^{c}COR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, —CR^{c}═CR^{a}R^{b}, —CRC═CHR^{a}, —CRC═CR^{a}R^{b}, —CCR^{a}, —SH, —SR^{a}, —S(O)R^{a}, —S(O)₂R^{a}. R^{a}-R^{d} are each independently an alkyl group, aromatic group, non-aromatic heterocyclic group or —N(R^{a}R^{b}), taken together, form an optionally substituted non-aromatic heterocyclic group. The alkyl, aromatic and non-aromatic heterocyclic group represented by R^{a}-R^{d} and the non-aromatic heterocyclic group represented by —N(R^{a}R^{b}) are each optionally and independently substituted with one or more groups represented by R^{#}.

R^{#} is R⁺, —OR⁺, —O(haloalkyl), —SR⁺, —NO₂, —CN, —NCS, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R⁺, —C(O)R⁺, —C(O)N(R⁺)₂, —OC(O)R⁺, —OC(O)N(⁺)₂, —S(O)₂R⁺, —SO₂N(R⁺)₂, —S(O)R⁺, —NHSO₂N(⁺)₂, —NHSO₂R⁺, —C(═S)N(R⁺)₂, or —C(═NH)—N(R⁺)₂.

R⁺ is —H, a C1-C4 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine. Optionally, the group —N(R⁺)₂ is a non-aromatic heterocyclic group, provided that non-aromatic heterocyclic groups represented by R⁺and —N(R⁺)₂ that comprise a secondary ring amine are optionally acylated or alkylated.

Preferred substituents for a phenyl group, including phenyl groups represented by R₁-R₄, include C1-C4 alkyl, C1-C4 alkoxy, C1-C4 haloalkyl, C1-C4 haloalkoxy, phenyl, benzyl, pyridyl, —OH, —NH₂, —F, —Cl, —Br, —I, —NO₂ or —CN.

Preferred substituents for a cycloalkyl group, including cycloalkyl groups represented by R₁ and R₂, are alkyl groups, such as a methyl or ethyl groups.

In one embodiment, Y in Structural Formula I is a covalent bond, —C(R₅R₆)—, —(CH₂CH₂)—, trans-(CH═CH)—, cis-(CH═CH)— or —(C≡C)— group, preferably —C(R₅R₆)—. R₁-R₄ are as described above for Structural Formula I. R₅ and R₆ are each independently —H, an aliphatic or substituted aliphatic group, or R₅ is —H and R₆ is an optionally substituted aryl group, or, R₅ and R₆, taken together, are an optionally substituted C2-C6 alkylene group. The pharmaceutically acceptable cation is as described in detail below.

In specific embodiments, Y taken together with both >C=Z groups to which it is bonded, is an optionally substituted aromatic group. In this instance, certain bis(thio-hydrazide amides) are represented by Structural Formula II: 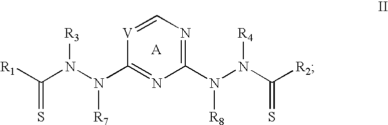
wherein Ring A is substituted or unsubstituted and V is —CH— or —N—. The other variables in Structural Formula II are as described herein for Structural Formula I or III.

In particular embodiments, the bis(thio-hydrazide amides) are represented by Structural Formula III:

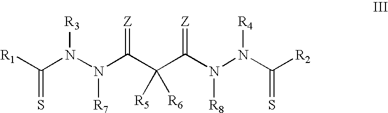

R₁-R₈ and the pharmaceutically acceptable cation are as described above for Structural Formula I.

In Structural Formulas I-III, R₁ and R₂ are the same or different and/or R₃ and R₄ are the same or different; preferably, R₁ and R₂ are the same and R₃ and R₄ are the same. In Structural Formulas I and III, Z is preferably O. Typically in Structural Formulas I and III, Z is O; R₁ and R₂ are the same; and R₃ and R₄ are the same. More preferably, Z is O; R₁ and R₂ are the same; R₃ and R₄ are the same, and R₇ and R₈ are the same.

In other embodiments, the bis(thio-hydrazide amides) are represented by Structural Formula III: R₁ and R₂ are each an optionally substituted aryl group, preferably an optionally substituted phenyl group; R₃ and R₄ are each an optionally substituted aliphatic group, preferably an alkyl group, more preferably, methyl or ethyl; and R₅ and R₆ are as described above, but R₅ is preferably —H and R₆ is preferably —H, an aliphatic or substituted aliphatic group.

Alternatively, R₁ and R₂ are each an optionally substituted aryl group; R₃ and R₄ are each an optionally substituted aliphatic group; R₅ is —H; and R₆ is —H, an aliphatic or substituted aliphatic group. Preferably, R₁ and R₂ are each an optionally substituted aryl group; R₃ and R₄ are each an alkyl group; and R₅ is —H and R₆ is —H or methyl. Even more preferably, R₁ and R₂ are each an optionally substituted phenyl group; R₃ and R₄ are each methyl or ethyl; and R₅ is —H and R₆ is —H or methyl. Suitable substituents for an aryl group represented by R₁ and R₂ and an aliphatic group represented by R₃, R₄ and R₆ are as described below for aryl and aliphatic groups.

In another embodiment, the bis(thio-hydrazide amides) are represented by Structural Formula III: R₁ and R₂ are each an optionally substituted aliphatic group, preferably a C3-C8 cycloalkyl group optionally substituted with at least one alkyl group, more preferably cyclopropyl or 1-methylcyclopropyl; R₃ and R₄ are as described above for Structural Formula I, preferably both an optionally substituted alkyl group; and R₅ and R₆ are as described above, but R₅ is preferably —H and R₆ is preferably —H, an aliphatic or substituted aliphatic group, more preferably —H or methyl.

Alternatively, the bis(thio-hydrazide amides) are represented by Structural Formula III: R₁ and R₂ are each an optionally substituted aliphatic group; R₃ and R₄ are as described above for Structural Formula I, preferably both an optionally substituted alkyl group; and R₅ is —H and R₆ is —H or an optionally substituted aliphatic group. Preferably, R₁ and R₂ are both a C3-C8 cycloalkyl group optionally substituted with at least one alkyl group; R₃ and R₄ are both as described above for Structural Formula I, preferably an alkyl group; and R₅ is —H and R₆ is —H or an aliphatic or substituted aliphatic group. More preferably, R₁ and R₂ are both a C3-C8 cycloalkyl group optionally substituted with at least one alkyl group; R₃ and R₄ are both an alkyl group; and R₅ is —H and R₆ is —H or methyl. Even more preferably, R₁ and R₂ are both cyclopropyl or 1-methylcyclopropyl; R₃ and R₄ are both an alkyl group, preferably methyl or ethyl; and R₅ is —H and R₆ is —H or methyl.

In specific embodiments, the bis(thio-hydrazide amides) are represented by Structural Formula IV:

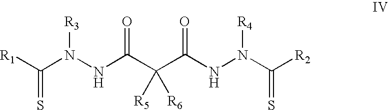

wherein: R₁ and R₂ are both phenyl, R₃ and R₄ are both methyl, and R₅ and R₆ are both —H; R₁ and R₂ are both phenyl, R₃ and R₄ are both ethyl, and R₅ and R₆ are both —H; R₁ and R₂ are both 4-cyanophenyl, R₃ and R₄ are both methyl, R₅ is methyl, and R₆ is —H; R₁ and R₂ are both 4-methoxyphenyl, R₃ and R₄ are both methyl, and R₅ and R₆ are both —H; R₁ and R₂ are both phenyl, R₃ and R₄ are both methyl, R₅ is methyl, and R₆ is —H; R₁ and R₂ are both phenyl, R₃ and R₄ are both ethyl, R₅ is methyl, and R₆ is —H; R₁ and R₂ are both 4-cyanophenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both ₃-cyanophenyl, R₃ and R₄ are both methyl, and R₅ and R₆ are both —H; R₁ and R₂ are both ₃-fluorophenyl, R₃ and R₄ are both methyl, and R₅ and R₆ are both —H; R₁ and R₂ are both 4-chlorophenyl, R₃ and R₄ are both methyl, R₅ is methyl, and R₆ is —H; R₁ and R₂ are both 2-dimethoxyphenyl, R₃ and R₄ are both methyl, and R₅ and R₆ are both —H; R₁ and R₂ are both 3-methoxyphenyl, R₃ and R₄ are both methyl, and R₅ and R₆ are both —H; R₁ and R₂ are both 2,3-dimethoxyphenyl, R₃ and R₄ are both methyl, and R₅ and R₆ are both —H; R₁ and R₂ are both 2,3-dimethoxyphenyl, R₃ and R₄ are both methyl, R₅ is methyl, and R₆ is —H; R₁ and R₂ are both 2,5-difluorophenyl, R₃ and R₄ are both methyl, and R₅ and R₆ are both —H; R₁ and R₂ are both 2,5-difluorophenyl, R₃ and R₄ are both methyl, R₅ is methyl, and R₆ is —H; R₁ and R₂ are both 2,5-dichlorophenyl, R₃ and R₄ are both methyl, and R₅ and R₆ are both —H; R₁ and R₂ are both 2,5-dimethylphenyl, R₃ and R₄ are both methyl, and R₅ and R₆ are both —H; R₁ and R₂ are both 2,5-dimethoxyphenyl, R₃ and R₄ are both methyl, and R₅ and R₆ are both —H; R₁ and R₂ are both phenyl, R₃ and R₄ are both methyl, and R₅ and R₆ are both —H; R₁ and R₂ are both 2,5-dimethoxyphenyl, R₃ and R₄ are both methyl, R₅ is methyl, and R₆ is —H; R₁ and R₂ are both cyclopropyl, R₃ and R₄ are both methyl, and R₅ and R₆ are both —H; R₁ and R₂ are both cyclopropyl, R₃ and R₄ are both ethyl, and R₅ and R₆ are both —H; R₁ and R₂ are both cyclopropyl, R₃ and R₄ are both methyl, R₅ is methyl, and R₆ is —H; R₁ and R₂ are both 1-methylcyclopropyl, R₃ and R₄ are both methyl, and R₅ and R₆ are both —H; R₁ and R₂ are both 1-methylcyclopropyl, R₃ and R₄ are both methyl, R₅ is methyl and R₆ is —H; R₁ and R₂ are both 1-methylcyclopropyl, R₃ and R₄ are both methyl, R₅ is ethyl, and R₆ is —H; R₁ and R₂ are both 1-methylcyclopropyl, R₃ and R₄ are both methyl, R₅ is n-propyl, and R₆ is —H; R₁ and R₂ are both 1-methylcyclopropyl, R₃ and R₄ are both methyl, and R₅ and R₆ are both methyl; R₁ and R₂ are both 1-methylcyclopropyl, R₃ and R₄ are both ethyl, and R₅ and R₆ are both —H; R₁ and R₂ are both 1-methylcyclopropyl, R₃ is methyl, R₄ is ethyl, and R₅ and R₆ are both —H; R₁ and R₂ are both 2-methylcyclopropyl, R₃ and R₄ are both methyl, and R₅ and R₆ are both —H; R₁ and R₂ are both 2-phenylcyclopropyl, R₃ and R₄ are both methyl, and R₅ and R₆ are both —H; R₁ and R₂ are both 1-phenylcyclopropyl, R₃ and R₄ are both methyl, and R₅ and R₆ are both —H; R₁ and R₂ are both cyclobutyl, R₃ and R₄ are both methyl, and R₅ and R₆ are both —H; R₁ and R₂ are both cyclopentyl, R₃ and R₄ are both methyl, and R₅ and R₆ are both —H; R₁ and R₂ are both cyclohexyl, R₃ and R₄ are both methyl, and R₅ and R₆ are both —H; R₁ and R₂ are both cyclohexyl, R₃ and R₄ are both phenyl, and R₅ and R₆ are both —H; R₁ and R₂ are both methyl, R₃ and R₄ are both methyl, and R₅ and R₆ are both —H; R₁ and R₂ are both methyl, R₃ and R₄ are both t-butyl, and R₅ and R₆ are both —H; R₁ and R₂ are both methyl, R₃ and R₄ are both phenyl, and R₅ and R₆ are both —H; R₁ and R₂ are both t-butyl, R₃ and R₄ are both methyl, and R₅ and R₆ are both —H; R₁ and R₂ are ethyl, R₃ and R₄ are both methyl, and R₅ and R₆ are both —H; or R₁ and R₂ are both n-propyl, R₃ and R₄ are both methyl, and R₅ and R₆ are both —H.

In specific embodiments, the bis(thio-hydrazide amides) are represented by Structural Formula V: 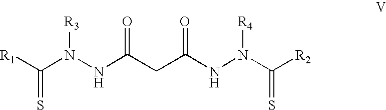
wherein: R₁ and R₂ are both phenyl, and R₃ and R₄ are both o-CH₃-phenyl; R₁ and R₂ are both o-CH₃C(O)O-phenyl, and R₃ and R₄ are phenyl; R₁ and R₂ are both phenyl, and R₃ and R₄ are both methyl; R₁ and R₂ are both phenyl, and R₃ and R₄ are both ethyl; R₁ and R₂ are both phenyl, and R₃ and R₄ are both n-propyl; R₁ and R₂ are both p-cyanophenyl, and R₃ and R₄ are both methyl; R₁ and R₂ are both p-nitro phenyl, and R₃ and R₄ are both methyl; R₁ and R₂ are both 2,5-dimethoxyphenyl, and R₃ and R₄ are both methyl; R₁ and R₂ are both phenyl, and R₃ and R₄ are both n-butyl; R₁ and R₂ are both p-chlorophenyl, and R₃ and R₄ are both methyl; R₁ and R₂ are both 3-nitrophenyl, and R₃ and R₄ are both methyl; R₁ and R₂ are both 3-cyanophenyl, and R₃ and R₄ are both methyl; R₁ and R₂ are both 3-fluorophenyl, and R₃ and R₄ are both methyl; R₁ and R₂ are both 2-furanyl, and R₃ and R₄ are both phenyl; R₁ and R₂ are both 2-methoxyphenyl, and R₃ and R₄ are both methyl; R₁ and R₂ are both 3-methoxyphenyl, and R₃ and R₄ are both methyl; R₁ and R₂ are both 2,3-dimethoxyphenyl, and R₃ and R₄ are both methyl; R₁ and R₂ are both 2-methoxy-5-chlorophenyl, and R₃ and R₄ are both ethyl; R₁ and R₂ are both 2,5-difluorophenyl, and R₃ and R₄ are both methyl; R₁ and R₂ are both 2,5-dichlorophenyl, and R₃ and R₄ are both methyl; R₁ and R₂ are both 2,5-dimethylphenyl, and R₃ and R₄ are both methyl; R₁ and R₂ are both 2-methoxy-5-chlorophenyl, and R₃ and R₄ are both methyl; R₁ and R₂ are both 3,6-dimethoxyphenyl, and R₃ and R₄ are both methyl; R₁ and R₂ are both phenyl, and R₃ and R₄ are both 2-ethylphenyl; R₁ and R₂ are both 2-methyl-5-pyridyl, and R₃ and R₄ are both methyl; or R₁ is phenyl; R₂ is 2,5-dimethoxyphenyl, and R₃ and R₄ are both methyl; R₁ and R₂ are both methyl, and R₃ and R₄ are both p-CF₃-phenyl; R₁ and R₂ are both methyl, and R₃ and R₄ are both o-CH₃-phenyl; R₁ and R₂ are both —CH₂)₃COOH; and R₃ and R₄ are both phenyl; R₁ and R₂ are both represented by the following structural formula: 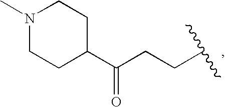
and R₃ and R₄ are both phenyl; R₁ and R₂ are both n-butyl, and R₃ and R₄ are both phenyl; R₁ and R₂ are both n-pentyl, R₃ and R₄ are both phenyl; R₁ and R₂ are both methyl, and R₃ and R₄ are both 2-pyridyl; R₁ and R₂ are both cyclohexyl, and R₃ and R₄ are both phenyl; R₁ and R₂ are both methyl, and R₃ and R₄ are both 2-ethylphenyl; R₁ and R₂ are both methyl, and R₃ and R₄ are both 2,6-dichlorophenyl; R₁-R₄ are all methyl; R₁ and R₂ are both methyl, and R₃ and R₄ are both t-butyl; R₁ and R₂ are both ethyl, and R₃ and R₄ are both methyl; R₁ and R₂ are both t-butyl, and R₃ and R₄ are both methyl; R₁ and R₂ are both cyclopropyl, and R₃ and R₄ are both methyl; R₁ and R₂ are both cyclopropyl, and R₃ and R₄ are both ethyl; R₁ and R₂ are both 1-methylcyclopropyl, and R₃ and R₄ are both methyl; R₁ and R₂ are both 2-methylcyclopropyl, and R₃ and R₄ are both methyl; R₁ and R₂ are both 1-phenylcyclopropyl, and R₃ and R₄ are both methyl; R₁ and R₂ are both 2-phenylcyclopropyl, and R₃ and R₄ are both methyl; R₁ and R₂ are both cyclobutyl, and R₃ and R₄ are both methyl; R₁ and R₂ are both cyclopentyl, and R₃ and R₄ are both methyl; R₁ is cyclopropyl, R₂ is phenyl, and R₃ and R₄ are both methyl.

Preferred examples of bis(thio-hydrazide amides) include Compounds (1)-(18) and pharmaceutically acceptable salts and solvates thereof:

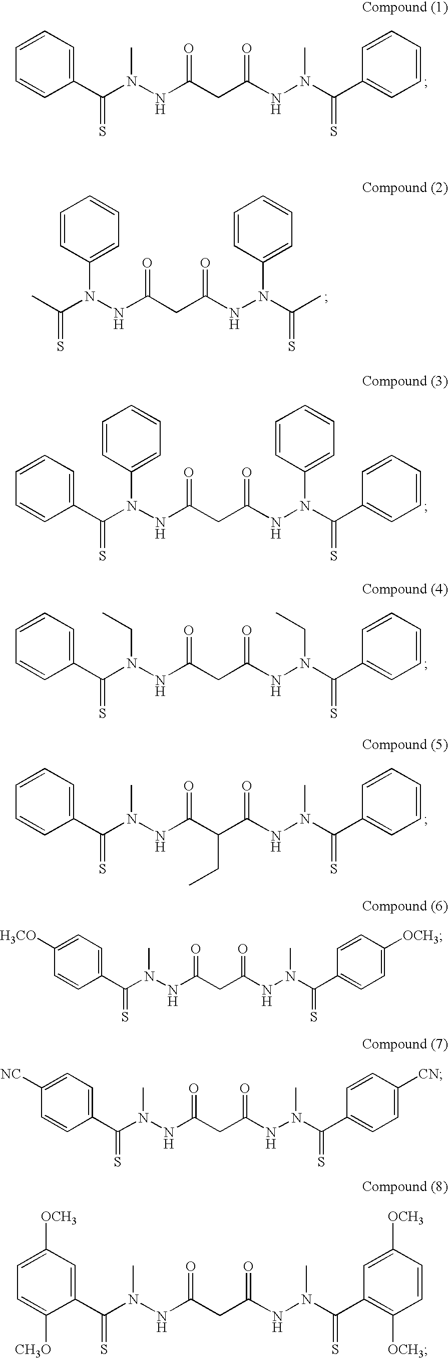

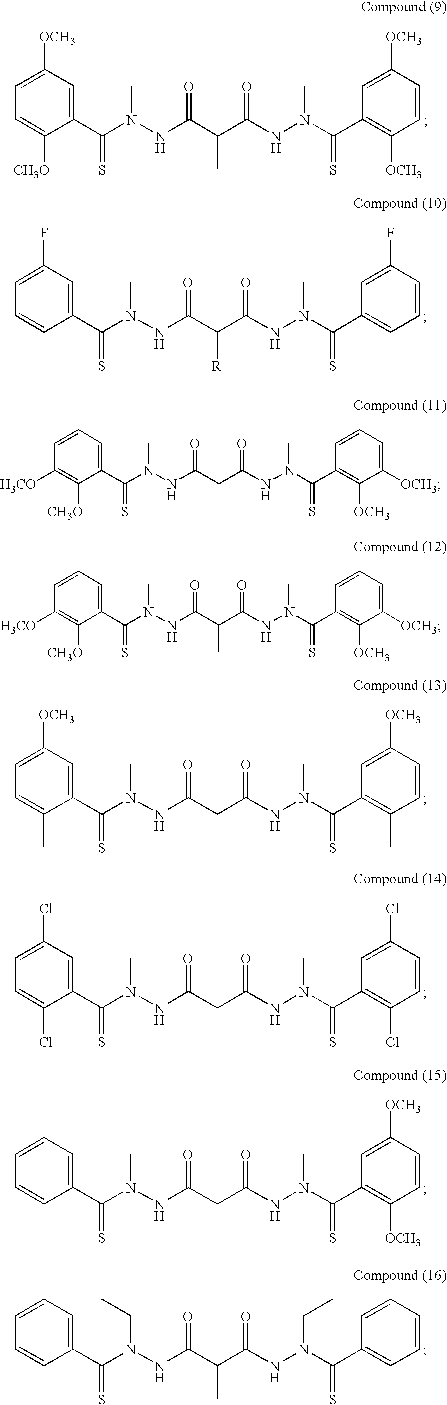

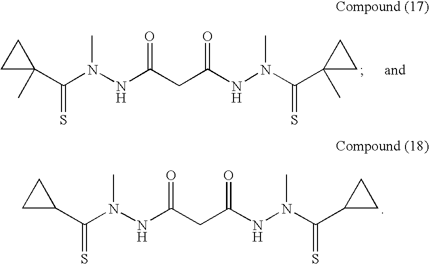

Preferred examples of bis(thio-hydrazide amides) include Compounds (1), (17), and (18) and pharmaceutically acceptable salts and solvates thereof.

As used herein, the term “bis(thio-hydrazide amides)” and references to the Structural Formulas of this invention also include pharmaceutically acceptable salts and solvates of these compounds and Structural Formulas.

As used herein, the terms “proliferative disorder”, “hyperproliferative disorder,” and “cell proliferation disorder” are used interchangeably to mean a disease or medical condition involving pathological growth of cells. Such disorders include cancer, except where specifically excluded.

Non-cancerous proliferative disorders include smooth muscle cell proliferation, systemic sclerosis, cirrhosis of the liver, adult respiratory distress syndrome, idiopathic cardiomyopathy, lupus erythematosus, retinopathy, e.g., diabetic retinopathy or other retinopathies, cardiac hyperplasia, reproductive system associated disorders such as benign prostatic hyperplasia and ovarian cysts, pulmonary fibrosis, endometriosis, fibromatosis, harmatomas, lymphangiomatosis, sarcoidosis, desmoid tumors and the like.

Smooth muscle cell proliferation includes proliferative vascular disorders, for example, intimal smooth muscle cell hyperplasia, restenosis and vascular occlusion, particularly stenosis following biologically- or mechanically-mediated vascular injury, e.g., vascular injury associated with balloon angioplasty or vascular stenosis. Moreover, intimal smooth muscle cell hyperplasia can include hyperplasia in smooth muscle other than the vasculature, e.g., hyperplasia in bile duct blockage, in bronchial airways of the lung in asthma patients, in the kidneys of patients with renal interstitial fibrosis, and the like.

Non-cancerous proliferative disorders also include hyperproliferation of cells in the skin such as psoriasis and its varied clinical forms, Reiter's syndrome, pityriasis rubra pilaris, and hyperproliferative variants of disorders of keratinization (e.g., actinic keratosis, senile keratosis), scleroderma, and the like.

As used herein, a “medical device” is a device used in therapy that is located at a site on or in a subject for an extended period of time (e.g., a time period of at least an hour, 6 hours, a day, three days, a week, two weeks, a month, two months, six months, a year, or longer than 2 years). Capsules and tablets for oral administration are specifically excluded from the term “medical device”.

In one embodiment, the medical device is located at a treatment site. As used herein, “located at the treatment site” means the medical device either physically contacts the treatment site, or is located in such close proximity to the treatment site that it can release the bis(thio-hydrazide amide) so that the concentration at the treatment site can be greater than the concentration achievable by systemic (intravenous) administration of the same amount of the bis(thio-hydrazide amide) by a ratio of at least about 2:1, generally at least about 10:1, typically about 50:1, more typically about 100:1 and preferably about 250:1. The device is located at the treatment site by surgical insertion or by mechanical or adhesive attachment. For example, a heart valve or stent can be surgically inserted, a cervical ring can be attached by mechanical force between the ring and the cervix, a transdermal patch can be attached to the skin by an adhesive, and the like.

In one embodiment, the medical device is a device used to treat a non-cancerous proliferative disorder and the medical device is not located at a treatment site. For example, a patch, comprising a bis(thio-hydrazide amide) and which adheres to the skin, can be used to treat an internal disorder, such as endometriosis or benign prostatic hyperplasia. In another embodiment, the medical device used to treat a non-cancerous proliferative disorder can be located at the treatment site. For example, a stent that comprises a bis(thio-hydrazide amide) can be surgically inserted at a site of vascular injury associated with balloon angioplasty to prevent or reduce restenosis at the site.

When medical device comprising a bis(thio-hydrazide amide) is used to treat a cancerous proliferative disorder, medical devices, such as syringes and IV drip lines, which are typically used for systemic administration, are specifically excluded. In addition, suppositories and inhalation device for administration of a bis(thio-hydrazide amide) are specifically excluded from the term “medical device” when a cancerous proliferative disorder is being treated. In another embodiment, the medical device is used to treat a cancerous proliferative disorder and the medical device is located at a treatment site. For example, a cervical ring comprising a bis(thio-hydrazide amide) can be used to treat tumors located in the cervics; or a patch or adhesive bandage comprising a bis(thio-hydrazide amide) can be used to cover the site at which a melanoma has been remove to prevent or reduce tumor regrowth.

In one embodiment, a “medical device” is a device that substantially retains its mass over the duration of treatment. “Substantially maintains” means the mass is retained (e.g., a percentage of the original mass is retained of at least about 75%, at least about 90%, at least about 95%, at least about 97%, or at least about 99%) except for changes in mass due to release of the bis(thio-hydrazide amide) and accompanying coatings, excipients, and the like. For example, a stent can be a metal mesh that is implanted to hold open a blocked blood vessel; a pacemaker can include a controller/electrode that delivers a signal to the heart; a tissue augmentation implant, e.g. a breast implant, can be a filled balloon that supports tissue; and the like. Each of the preceeding can have the bis(thio-hydrazide amide) incorporated for release, e.g., in a coating. In the stent example, the mass and shape of the metal mesh portion of the stent can be substantially retained after implantation, while the coating can lose mass as the bis(thio-hydrazide amide) is released.

As used herein, a “treatment site” is the site in the subject that is in need of treatment for cell proliferation. The treatment site can be the same or different from a treatment objective of the medical device. For example, when a pacemaker (e.g., including a controller implanted under the skin and an electrode extending therefrom to the heart) is implanted to stimulate the heart via an electrode, the treatment objective of the device can be the heart while the treatment site at risk of cell proliferation can be at any portion of the device contacting the subject, e.g., at the electrode contacting the heart, the site of implantation of the controller, and the like.

A treatment site can develop at sites in a subject (e.g., vascular sites subject to angioplasty and stent insertion) which are in contact with a synthetic material or a medical device. Treatment sites can also develop at non-vascular sites, for example at sites where a therapeutic effect can be achieved by inhibiting cell proliferation. Examples include other sites subject to pathological cell hyperproliferation, for example, proliferation of cells in smooth muscle, e.g. smooth muscle cell proliferation in the bile duct leading to blockage; sites of implantation of any medical device wherein the site is at risk of excess cell growth leading to formation of scars, lesions, adhesions; and the like. A treatment site can also be at or in the vicinity of a malignant growth. Because bis(thio-hydrazide amides) are known anti-cancer agents and Taxol™ enhancers (U.S. Publication Nos. 2003/0045518 and 2003/0119914, both entitled “Synthesis of Taxol Enhancers” and also co-pending U.S. application Ser. No. 10/758,589, entitled “Treatment for Cancers”; the entire teachings of these applications are incorporated herein by reference), delivering bis(thio-hydrazide amides) directly to or in the vicinity of a cancer can be a particularly effective method of treatment.

A treatment site can develop as the result of biologically or mechanically-mediated injury. As used herein, “mechanically mediated injury” is tissue damage caused by the application of mechanical force. For example, a mechanically mediated injury can be the damage caused by surgical insertion of a medical device such as the damage caused to vasculature by inflation of an angioplasty balloon, and the like. As used herein, “biologically mediated injury” is tissue damage caused by a disease or disorder, for example, damage caused by bacterial agents, inflammation, and the like.

When a medical device is used, the device can be located at the treatment site for a time longer than the duration of treatment for cell proliferation, for example, in a stent intended to remain in a subject indefinitely, the stent can have a coating that begins releasing the bis(thio-hydrazide amide) at implantation and continues until the coating can be depleted, e.g., a duration of treatment for cell proliferation of several weeks, while the stent can remain in the subject indefinitely, e.g., months or years. The duration of treatment can be extended over multiple medical devices, for example, when the device is a transdermal patch, a first patch at a treatment site can be replaced with a second patch as the first patch becomes depleted in the bis(thio-hydrazide amide).

In one embodiment, the compounds of the present invention may be used topically. In such cases, the compounds may be formulated as a solution, gel, lotion, cream or ointment in a pharmaceutically acceptable form. Actual methods for preparing these, and other, topical pharmaceutical compositions are known or apparent to those skilled in the art and are described in detail in, for example, Remington's Pharmaceutical Sciences, 16^{th} and 18^{th} eds., Mack Publishing Company, Easton, Pa., 1980-1990).

In some embodiments of the invention, the mode of administration is by a medical device that releases the bis(thio-hydrazide amide) in vivo, e.g., the device includes a reservoir, a coating composition, a controlled release polymer matrix, or the like which comprises the bis(thio-hydrazide amide) and can release the bis(thio-hydrazide amide) in vivo. Details of releasing compounds in vivo are known in the art; see, for example, Baker, et al., “Controlled Release of Biological Active Agents”, John Wiley and Sons, 1986, the entire teachings of which are incorporated herein by reference.In one embodiment, the medical device is located at a treatment site in a subject in need of treatment.

Medical devices that are suitable for use in this invention include, but are not limited to, stents, e.g., coronary stents; peripheral stents; arterial and venous stents, stents for other vessels, e.g., the bile duct and urethra; catheters; arterio-venous grafts; by-pass grafts; drug delivery balloons used in the vasculature; sheaths for veins and arteries; GORE-TEX surgical prosthetics; artificial valves, artificial hearts, pacemakers, artificial joints, structural implants (pins, screws, plates, and the like), tooth implants, chochlear implants, breast implants, transdermal patches, adhesive bandages, vaginal sponges, cervical rings, ocular lenses, osmotic pumps, and the like.

In one embodiment, the stent comprises a reservoir, a coating composition, a controlled release polymer matrix, or the like which comprises the bis(thio-hydrazide amide) and can sustain the release of the bis(thio-hydrazide amide) in vivo. In a preferred embodiment, the stent is coated with a composition that comprises the bis(thio-hydrazide amide) and releases the bis(thio-hydrazide amide) in vivo. In one embodiment, surface contours can be placed on the medical device, for example, to allow for a reservoir to be placed in a stent to deliver the bis(thio-hydrazide amide).

In one embodiment, a stent is made of a metallic material such as stainless steel, tantalum, titanium alloys including nitinol and certain cobalt-chromium alloys. Alternatively, the stent may be made of a plastic material such as those described in U.S. Pat. Nos. 5,163,952 and 5,092,877, the entire teachings of each of these patents are incorporated herein by reference. In general, when the stent includes a coating composition, the composition may contain from about 0.01% to as high as about 80% or more of the bis(thio-hydrazide amide) by weight with respect to the total weight of the material and typically, the composition comprises between about 0.1% and about 45% of the bis(thio-hydrazide amide). Typically, the coating composition will have a thickness of between about 1 μm and about 1000 μm (e.g., between about 20 μm and about 200 μm, or between about 20 μm and 100 μm, or between about 30 μm and 75 μm, or between about 30 μm and 40 μm). Specific embodiments of the present invention include those designed to elute the bis(thio-hydrazide amide) over a period of weeks or months.

In one embodiment, the medical device is a transdermal patch. For example, patch suitable for use with the invention includes a layer comprising a bis(thio-hydrazide amide); typically, the patch can include a backing layer, e.g., to protect the layer comprising the compound. The patch may comprise an adhesive means for securing to the surface of the skin or mucosa. In a specific embodiment, a cover layer is also present to protect the layer comprising the bis(thio-hydrazide amide). In one embodiment, the patch can be located at a treatment site by adhesive attachment, where the bis(thio-hydrazide amide) can be released in vivo to the skin surface over time. In another embodiment, the patch is not located at the treatment site. The structural component of such patches, e.g., typically a biocompatible, non-biodegradeable backing layer, is typically intended to remain for the duration of treatment while a controlled release polymer matrix releases the bis(thio-hydrazide amide) to the subject's skin, after which the patch can be discarded, and (if indicated) another patch applied.

Patches that are suitable for use in this invention include, for example, a matrix type patch; a reservoir type patch; a monolithic drug-in-adhesive type patch; a multi-laminate drug-in-adhesive type patch; and the like. These patches are well known in the art; see, for example, Ghosh, T. K.; Pfister, W. R.; Yum, S. I. Transdermal and Topical Drug Delivery Systems, Interpharm Press, Inc. p. 249-297, the entire teachings of which are incorporated herein by reference. One of ordinary skill in the art can determine other patches which can be employed in the present invention.

In one embodiment, a patch can be designed to adhere to a mucous membrane surface of the subject, e.g., sublingual or buccal membrane of the oral cavity, and the like. Typically, such a patch will include a mucoadhesive that has been loaded with the bis(thio-hydrazide amide). In one embodiment, the bis(thio-hydrazide amide) is loaded into the adhesive by equilibrium swelling of the adhesive in a solution containing the bis(thio-hydrazide amide). Examples of typical mucosal adhesives are described in Nagai, J. Control. Rel. (1985), 2:121-134 and in Nagai, et al., Pharm. Int. (1985), 196-200; te entire teachings of these documents is incorporated herein by reference. In one embodiment, the medical device can be a bandage that includes an adhesive that comprises the bis(thio-hydrazide amide). The adhesive bandage can be located at the treatment site, e.g., the skin of the subject.

In another embodiment, the medical device is a vaginal delivery device, such as a vaginal sponge or a cervical ring which comprises the bis(thio-hydrazide amide) and releases it in vivo. The ring, for example, can be located at the treatment site by mechanical force of the ring at the area of the cervix. The bis(thio-hydrazide amide) can be released over a portion of that time beginning at about the time of insertion.

In one embodiment, the medical device is an ocular delivery device, e.g., an ocular lens, which comprises the bis(thio-hydrazide amide) and releases it in vivo.

In one embodiment, the medical device is an implantable osmotic pump which can be used as a means for continuous infusion of the bis(thio-hydrazide amide) in vivo. Such osmotic pumps can allow for targeted delivery to a localized treatment site.

Further, the bis(thio-hydrazide amides) described herein can be combined with any medical device, wherein placement of the device at a treatment site in the subject can place that site at risk of pathological cell proliferation in response to tissue injury associated with placement of the device (e.g., formation of scars, lesions, adhesions, and the like.)

The bis(thio-hydrazide amides) described herein can be applied to or incorporated in the medical devices, typically in combination with a polymeric compound, for example, a polymer, a polymeric coating composition, and the like. Incorporation of the compound or drug into a polymeric coating composition of the medical device can be carried out by any conventional means, for example, forming a premixed composition of the compound and a polymer and forming the device, forming a premixed composition of the compound and a polymer and then coating a device, precoating a device with the polymer and then contacting the polymer coating composition with the compound, whereby the compound is absorbed into or onto the polymer; and the like. The compound, optionally in combination with the polymer, can be applied by any conventional means such as dip coating, roll coating, spray coating, spin coating, vapor condensation, and the like.

Drug release surface coating compositions on medical devices in accordance with the present invention can release drugs over a period of time from days to months and can be used, for example, to inhibit smooth muscle cell migration and proliferation and to inhibit hyperplasia and restenosis. As such, they can be used for chronic patency after an angioplasty or stent placement. It is further anticipated that the need for a second angioplasty procedure may be obviated in a significant percentage of patients in which a repeat procedure would otherwise be necessary. Such antiproliferative applications can include not only cardiovascular but any tubular vessel that stents are placed including urologic, pulmonary and gastrointestinal.

Various combinations of polymer coating composition materials can be coordinated with the medical device, e.g., stent, and the bis(thio-hydrazide amide) to produce a combination which is compatible at the implant site of interest and controls the release of the compound over a desired time period. In a particular embodiment, coating polymers include silicones (poly siloxanes), polyurethanes, thermoplastic elastomers in general, ethylene vinyl acetate copolymers, polyolefin rubbers, EPDM rubbers, and combinations thereof.

A suitable coating composition can comprise any polymeric material with which the bis(thio-hydrazide amides) can be combined to form a coating, e.g., a polymer in which a bis(thio-hydrazide amide) is soluble or dispersable. The coating composition can serve as a controlled release vehicle for the therapeutic agent to be delivered at the site of a lesion, and can be selected such that the bis(thio-hydrazide amide) can be released at a desired rate in vivo. The coating composition can be polymeric and can further be hydrophilic, hydrophobic, biodegradable, or non-biodegradable. As used herein “polymer” has the meaning commonly afforded the term. Example are homopolymers, co-polymers (including block copolymers and graft copolymers), dendritic polymers, crosslinked polymers and the like. Suitable polymers include synthetic and natural polymers (e.g. polysaccharides, peptides) as well as polymers prepared by condensation, addition and ring opening polymerizations. Also included are rubbers, fibers and plastics. Polymers can be hydrophilic, amphiphilic or hydrophobic. In one aspect, the polymers of the present invention are non-peptide polymers.

The material for the polymeric coating composition can be selected from the group consisting of polycarboxylic acids, cellulosic polymers, gelatin, polyvinylpyrrolidone, maleic anhydride polymers, polyamides, polyvinyl alcohols, polyethylene oxides, glycosaminoglycans, polysaccharides, polyesters, polyurethanes, silicones, polyorthoesters, polyanhydrides, polycarbonates, polypropylenes, polylactic acids, polyglycolic acids, polycaprolactones, polyhydroxybutyrate valerates, polyacrylamides, polyethers, and mixtures and copolymers of the foregoing. Coating compositions prepared from polymeric dispersions such as polyurethane dispersions (BAYHYDROL, etc.) and acrylic acid latex dispersions can also be employed.

Biodegradable polymers that can employed in the coating composition include polymers such as poly(L-lactic acid), poly(DL-lactic acid), polycaprolactone, poly(hydroxy butyrate), polyglycolide, poly(diaxanone), poly(hydroxy valerate), polyorthoester; copolymers such as poly (lactide-co-glycolide), polyhydroxy(butyrate-co-valerate), polyglycolide-co-trimethylene carbonate; polyanhydrides; polyphosphoester; polyphosphoester-urethane; polyamino acids; polycyanoacrylates; biomolecules such as fibrin, fibrinogen, cellulose, starch, collagen and hyaluronic acid; and mixtures of the foregoing. Biostable materials that can be employed in the coating composition include polymers such as polyurethane, silicones, polyesters, polyolefins, polyamides, polycaprolactam, polyimide, polyvinyl chloride, polyvinyl methyl ether, polyvinyl alcohol, acrylic polymers and copolymers, polyacrylonitrile, polystyrene copolymers of vinyl monomers with olefins (such as styrene acrylonitrile copolymers, ethylene methyl methacrylate copolymers, ethylene vinyl acetate), polyethers, rayons, cellulosics (such as cellulose acetate, cellulose nitrate, cellulose propionate, etc.), parylene and derivatives thereof; and mixtures and copolymers of the foregoing.

Another polymer that can be that can be employed in the coating composition is poly(MPC_{w}:LAMₓ:HPMA_{y}:TSMA_{z}) where w, x, y, and z represent the molar ratios of monomers used in the feed for preparing the polymer and MPC represents the unit 2-methacryoyloxyethylphosphorylcholine, LMA represents the unit lauryl methacrylate, HPMA represents the unit 2-hydroxypropyl methacrylate, and TSMA represents the unit 3-trimethoxysilylpropyl methacrylate. The coated medical device, e.g., stent, can be used to maintain patency of a blood vessel, e.g. coronary artery, previously occluded by thrombus and/or atherosclerotic plaque. The delivery of the bis(thio-hydrazide amides) described herein can reduce the rate of in-stent restenosis.

Particular polymers can be those which are water insoluble and hydrophilic, i.e. can form hydrogels. A hydrogel is a composition which can absorb large quantities of water. Polymers which can form hydrogels are generally more biocompatible than other polymers and can be used in devices which are inserted into, for example, vascular systems. Platelets and proteins typically deposit upon insertion of polymer into a treatment, e.g., vascular site and can initiate a cascade of events leading to restenosis or injury. This process can be slowed or eliminated with polymers that form hydrogels, resulting in reduced risk of protein deposition and platelet activation. Polymers which form hydrogels are typically crosslinked hydrophilic polymers. Further descriptions and examples of hydrogels are provided in Hydrogels and Biodegradable Polymers for Bioapplications, editors Attenbrite, Huang and Park, ACS Symposium Series, No. 627 (1996), U.S. Pat. Nos. 5,476,654, 5,498,613 and 5,487,898, the teachings of which are incorporated herein by reference. Examples of hydrogels include polyethylene hydroxides, polysaccharides and crosslinked polysaccharides.

A “controlled release polymer matrix,” as used herein, is a polymer combined with an active agent, such as a bis(thio-hydrazide amide), so that the active agent is released from the material in a predesigned manner. For example, the active agent may be released in a constant manner over a predetermined period of time, it may be released in a cyclic manner over a predetermined period of time, or an environmental condition or external event may trigger the release of the active agent, and the like. In one embodiment, the controlled release polymer matrix includes a polymer that is biologically degradable, chemically degradable, or both biologically and chemically degradable. In another embodiment, the controlled release polymer matrix includes a non-degradable polymer.

Examples of suitable polymers for a controlled release polymer matix include the polymers used for polymer coating compositions. In one embodiment, a controlled release polymer matrix is a coating. In another embodiment, the controlled release polymer matrix is solid component that forms part of the structure of the medical device. For example, a portion (e.g., about 1%, about 5%, about 10%, about 20% or about 50%) of the fibers that make up a vascular graft can be made of a controlled release polymer matrix.

As used herein, “biodegradable” polymers are those that, when introduced into a subject, are broken down by the cellular machinery (biologically degradable) and/or by a chemical process, such as hydrolysis, (chemically degradable) into components that the cells can either reuse or dispose of without significant toxic effect on the cells. In preferred embodiments, the degradable polymers and their degradation byproducts are biocompatible.

The term “biocompatible” polymer, as used herein, is intended to describe polymers that are generally not toxic to cells. Compounds are “biocompatible” if their addition to cells in vitro results in less than or equal to about 20% cell death and if they do not induce significant inflammation or other such significant adverse effects in vivo.

Biocompatible polymers can be categorized as biodegradable and non-biodegradable. As stated above, biodegradable polymers degrade in vivo as a function of chemical composition, method of manufacture, and implant structure. Synthetic and natural polymers can be used although synthetic polymers are preferred due to more uniform and reproducible degradation and other physical properties. Examples of synthetic biodegradable polymers include polyanhydrides, polyhydroxyacids such as polylactic acid, polyglycolic acids and copolymers thereof, polyesters, polyamides, polyorthoesters, and some polyphosphazenes. Examples of naturally occurring biodegradable polymers include proteins and polysaccharides such as collagen, hyaluronic acid, albumin and gelatin. A bis(thio-hydrazide amide) can be encapsulated within, throughout, and/or on the surface of the implant. The compound is released by diffusion, degradation of the polymer, or a combination thereof. There are two general classes of biodegradable polymers: those degrading by bulk erosion and those degrading by surface erosion. U.S. Patents that describe the use of polyanhydrides for controlled delivery of substances include U.S. Pat. No. 4,857,311, U.S. Pat. No. 4,888,176, and U.S. Pat. No. 4,789,724 to Domb and Langer. The entire teachings of these patents are incorporated herein by reference.

Non-biodegradable polymers remain intact in vivo for extended periods of time (e.g., at least about one or more years). Drug loaded into the non-biodegradable polymer matrix is released by diffusion through the polymer's micropore lattice in a sustained and predictable fashion, which can be tailored to provide a rapid or a slower release rate by altering the percent drug loading, porosity of the matrix, and implant structure. Ethylene-vinyl acetate copolymer (EVAc) is an example of a nonbiodegradable polymer that has been used as a local delivery system for proteins and other micromolecules, as reported by Langer, R., and J. Folkman, Nature (London) 263:797-799 (1976). Other non-biodegradable polymers include polyurethanes, polyacrylonitriles, and some polyphosphazenes.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (e.g., one or more therapeutic agents such as the bis(thio-hydrazide amide)). In specific embodiments, the terms “treat”, “treatment” and “treating” refer to the amelioration of at least one measurable physical parameter of a proliferative disorder, not necessarily discernible by the patient. In other embodiments the terms “treat”, “treatment” and “treating” refer to the inhibition of the progression of a proliferative disorder, either physically by, e.g., stabilization of a discernible symptom, physiologically by, e.g., stabilization of a physical parameter, or both. In other embodiments the terms “treat”, “treatment” and “treating” refer to the inhibition or reduction in the onset, development or progression of one or more symptoms associated with a proliferative disorder.

As used herein, the terms “prevent”, “prevention” and “preventing” refer to the reduction in the risk of acquiring or developing a given proliferative disorder, or the reduction or inhibition of the recurrence, onset or development of one or more symptoms of a given proliferative disorder. In a preferred embodiment, a compound of the invention is administered as a preventative measure to a patient, preferably a human, having a genetic predisposition to a proliferative disorder.

Another embodiment of the present invention is a method of treating a subject with cancer using a medical device having a reservoir, coating composition, or controlled release polymer matrix comprising the bis(thio-hydrazide amides) described herein. The cancer can be a multi-drug resistant cancer as described below. One or more additional anti-cancer drugs can optionally be co-administered with the compound (e.g., in the reservoir, coating composition or controlled release polymer matrix with the bis(thio-hydrazide amide) or coadminstered by any conventional means of drug administration). Examples of anti-cancer drugs are described more fully below. In one embodiment, the co-administered anti-cancer drug is an agent that stabilizes mictotubules, such as a member of the taxane family (e.g., Taxol™ or an analog of Taxol™).

As used herein, “treating a subject with a cancer,” or similar terms, includes achieving, partially or substantially, one or more of the following: arresting the growth or spread of a cancer, reducing the extent of a cancer (e.g., reducing size of a tumor or reducing the number of affected sites), inhibiting the growth rate of a cancer, and ameliorating or improving a clinical symptom or indicator associated with a cancer (such as tissue or serum components).

Cancers that can be treated or prevented by the methods of the present invention include, but are not limited to human sarcomas and carcinomas, e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, retinoblastoma; leukemias.e.g., acute lymphocytic leukemia and acute myelocytic leukemia (myeloblastic, promyelocytic, myelomonocytic, monocytic and erythroleukemia); chronic leukemia (chronic myelocytic (granulocytic) leukemia and chronic lymphocytic leukemia); and polycythemia vera, lymphoma (Hodgkin's disease and non-Hodgkin's disease), multiple myeloma, Waldenstrobm's macroglobulinemia, and heavy chain disease.

Other examples of leukemias include acute and/or chronic leukemias, e.g., lymphocytic leukemia (e.g., as exemplified by the p388 (murine) cell line), large granular lymphocytic leukemia, and lymphoblastic leukemia; T-cell leukemias, e.g., T-cell leukemia (e.g., as exemplified by the CEM, Jurkat, and HSB-2 (acute), YAC-1(murine) cell lines), T-lymphocytic leukemia, and T-lymphoblastic leukemia; B cell leukemia (e.g., as exemplified by the SB (acute) cell line), and B-lymphocytic leukemia; mixed cell leukemias, e.g., B and T cell leukemia and B and T lymphocytic leukemia; myeloid leukemias, e.g., granulocytic leukemia, myelocytic leukemia (e.g., as exemplified by the HL-60 (promyelocyte) cell line), and myelogenous leukemia (e.g., as exemplified by the K562(chronic)cell line); neutrophilic leukemia; eosinophilic leukemia; monocytic leukemia (e.g., as exemplified by the THP-1(acute) cell line); myelomonocytic leukemia; Naegeli-type myeloid leukemia; and nonlymphocytic leukemia. Other examples of leukemias are described in Chapter 60 of The Chemotherapy Sourcebook, Michael C. Perry Ed., Williams & Williams (1992) and Section 36 of Holland Frie Cancer Medicine 5th Ed., Bast et al. Eds., B.C. Decker Inc. (2000). The entire teachings of the preceding references are incorporated herein by reference.

In one embodiment, the disclosed method is believed to be particularly effective in treating subject with non-solid tumors such as multiple myeloma. In another embodiment, the disclosed method is believed to be particularly effective against T-leukemia (e.g., as exemplified by Jurkat and CEM cell lines); B-leukemia (e.g., as exemplified by the SB cell line); promyelocytes (e.g., as exemplified by the HL-60 cell line); uterine sarcoma (e.g., as exemplified by the MES—SA cell line); monocytic leukemia (e.g., as exemplified by the THP-1(acute) cell line); and lymphoma (e.g., as exemplified by the U937 cell line).

Some of the disclosed methods can be particularly effective at treating subjects whose cancer has become “multi-drug resistant”. A cancer which initially responded to an anti-cancer drug becomes resistant to the anti-cancer drug when the anti-cancer drug is no longer effective in treating the subject with the cancer. For example, many tumors will initially respond to treatment with an anti-cancer drug by decreasing in size or even going into remission, only to develop resistance to the drug. Drug resistant tumors are characterized by a resumption of their growth and/or reappearance after having seemingly gone into remission, despite the administration of increased dosages of the anti-cancer drug. Cancers that have developed resistance to two or more anti-cancer drugs are said to be “multi-drug resistant”. For example, it is common for cancers to become resistant to three or more anti-cancer agents, often five or more anti-cancer agents and at times ten or more anti-cancer agents.

When used to treat a non-cancerous proliferative disorder, the bis(thio-hydrazide amides) described herein can be administered as a monotherapy. Alternatively, the compound can be administered in combination with one or more additional agents that inhibits cell proliferation or provide other desirable benefits, for example, anticancer agents, immunosuppressants, and the like. Specific examples of suitable agents for use in combination with the compounds of this invention include members of the taxane family (e.g., Taxol™, Taxotere™, and Taxol™ analogs) rapamycin, rapamycin analogs, and the like.

Taxol™, also referred to as “paclitaxel”, is a well-known anti-cancer drug which acts by enhancing and stabilizing microtubule formation. Many analogs of Taxol™ are known, including Taxotere™, also referred to as “docetaxol”. Taxol™ and Taxotere™ have the respective structural formulas:

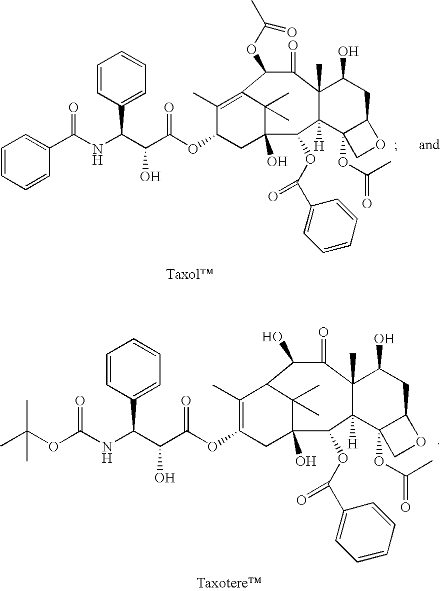

Taxol™ analogs, which have also been shown to have the ability to arrest cells in the G2-M phases due to stabilized microtubules, have the basic taxane skeleton as a common structure feature shown below in Structural Formula VI: 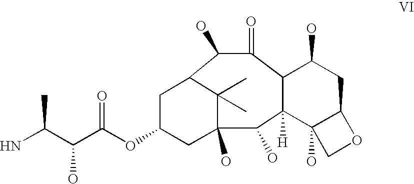
Double bonds have been omitted from the cyclohexane rings in the taxane skeleton represented by Structural Formula VI. It is to be understood that the basic taxane skeleton can include zero or one double bond in one or both cyclohexane rings, as indicated in the Taxol™ analogs and Structural Formulas VII and VIII below. A number of atoms have also been omitted from Structural Formula VI to indicate sites in which structural variation commonly occurs among Taxol™ analogs.

A wide variety of substituents can decorate the taxane skeleton without adversely affecting biological activity. Also, zero, one or both of the cyclohexane rings of a Taxol™ analog can have a double bond at the indicated positions. For example, substitution on the taxane skeleton with simply an oxygen atom indicates that hydroxyl, acyl, alkoxy or other oxygen-bearing substituent is commonly found at the site. It is to be understood that these and other substitutions on the taxane skeleton can be made without losing the ability to enhance and stabilize microtubule formation. Thus, the term “Taxol™ analog” is defined herein to mean a compound which has the basic Taxol™ skeleton and which stabilizes microtubule formation.

Typically, the Taxol™ analogs used herein are represented by Structural Formula VII or VIII:

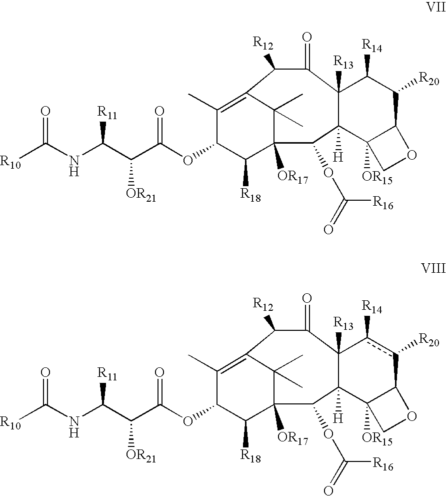

R₁₀ is an optionally substituted lower alkyl group, an optionally substituted phenyl group, —SR₁₉, —NHR₁₉ or —OR₁₉.

R₁₁ is an optionally substituted lower alkyl group, an optionally substituted aryl group.

R₁₂ is —H, —OH, lower alkyl, substituted lower alkyl, lower alkoxy, substituted lower alkoxy, —O—C(O)-(lower alkyl), —O—C(O)-(substituted lower alkyl), —O—CH₂—O-(lower alkyl) —S—CH₂—O-(lower alkyl).

R₁₃ is —H, —CH₃, or, taken together with R₁₄, —CH₂—.

R₁₄ is —H, —OH, lower alkoxy, —O—C(O)-(lower alkyl), substituted lower alkoxy, —O—C(O)-(substituted lower alkyl), —O—CH₂—O—P(O)(OH)₂, —O—CH₂—O-(lower alkyl), —O—CH₂—S-(lower alkyl) or, taken together with R₂₀, a double bond.

R₁₅—H, lower acyl, lower alkyl, substituted lower alkyl, alkoxymethyl, alkthiomethyl, —OC(O)—O(lower alkyl), —OC(O)—O(substituted lower alkyl), —OC(O)—NH(lower alkyl) or —OC(O)—NH(substituted lower alkyl).

R₁₆ is phenyl or substituted phenyl.

R₁₇ is —H, lower acyl, substituted lower acyl, lower alkyl, substituted, lower alkyl, (lower alkoxy)methyl or (lower alkyl)thiomethyl.

R₁₈—H, —CH₃ or, taken together with R₁₇ and the carbon atoms to which R₁₇ and R₁₈ are bonded, a five or six membered a non-aromatic heterocyclic ring.

R₁₉ is an optionally substituted lower alkyl group, an optionally substituted phenyl group.

R₂₀ is —H or a halogen.

R₂₁ is —H, lower alkyl, substituted lower alkyl, lower acyl or substituted lower acyl.

Preferably, the variables in Structural Formulas VII and VIII are defined as follows: R₁₀ is phenyl, tert-butoxy, —S—CH₂—CH—(CH₃)₂, —S—CH(CH₃)₃, —S—(CH₂)₃CH₃, —O—CH(CH₃)₃, —NH—CH(CH₃)₃, —CH═C(CH₃)₂ or para-chlorophenyl; R₁₁is phenyl, (CH₃)₂CHCH₂—, -2-furanyl, cyclopropyl or para-toluyl; R₁₂ is —H, —OH, CH₃CO— or —(CH₂)₂-N-morpholino; R₁₃ is methyl, or, R₁₃ and R₁₄, taken together, are —CH₂—;

R₁₄ is —H, —CH₂SCH₃ or —CH₂—O—P(O)(OH)₂; R₁₅ is CH₃CO—;

R₁₆ is phenyl; R₁₇—H, or, R₁₇ and R₁₈, taken together, are —O—CO—O—;

R₁₈ is —H; R₂₀ is —H or —F; and R₂, is —H, —C(O)—CHBr—(CH₂)₁₃—CH₃ or —C(O)—(CH₂)₁₄—CH₃; —C(O)—CH₂—CH(OH)—COOH, —C(O)—CH₂—O—C(O)—CH₂CH(NH₂)—CONH₂, —C(O)—CH₂—O—CH₂CH₂OCH₃ or —C(O)—O—C(O)—CH₂CH₃.
Specific examples of Taxol™ analogs include the following compounds:

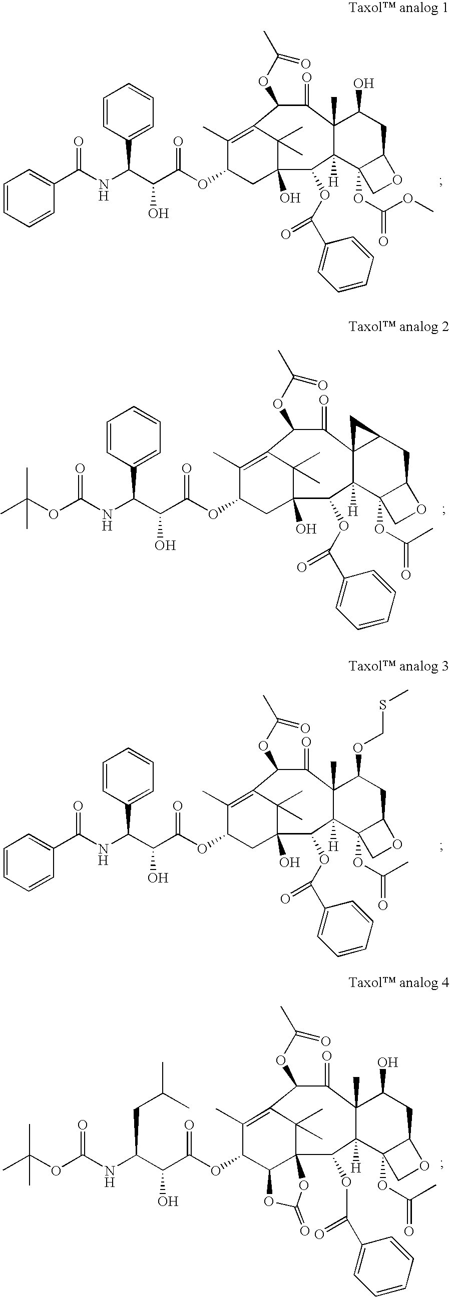

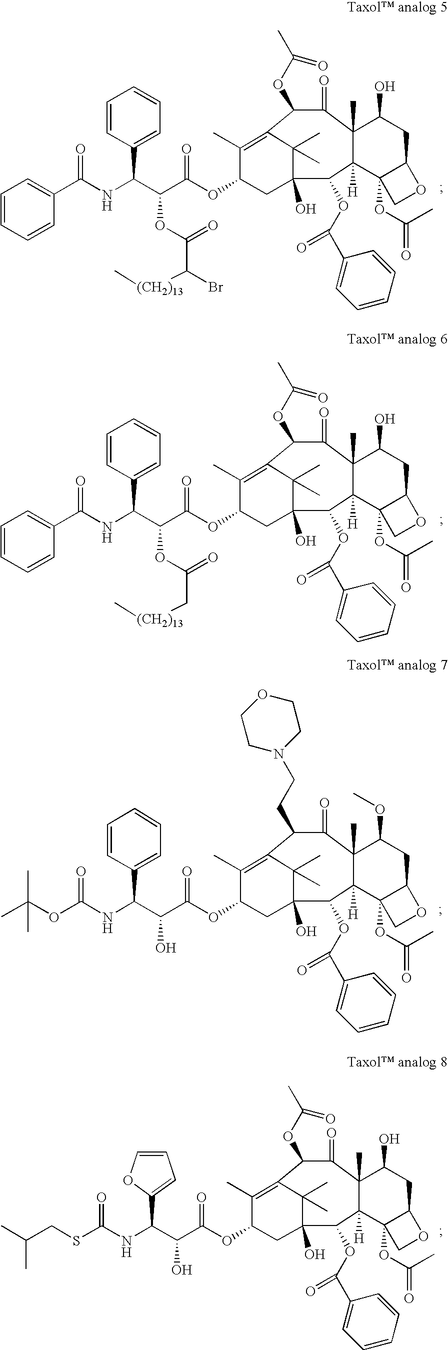

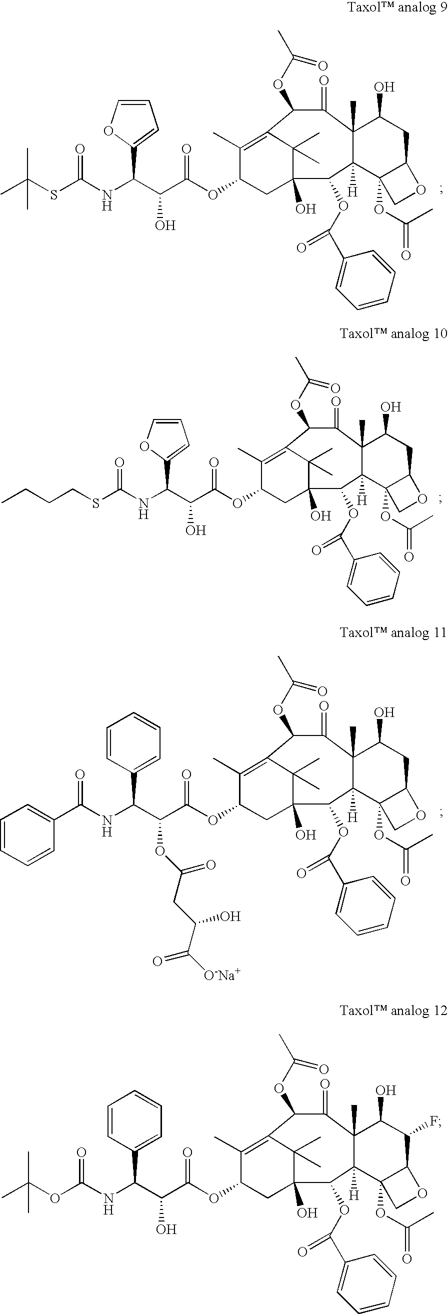

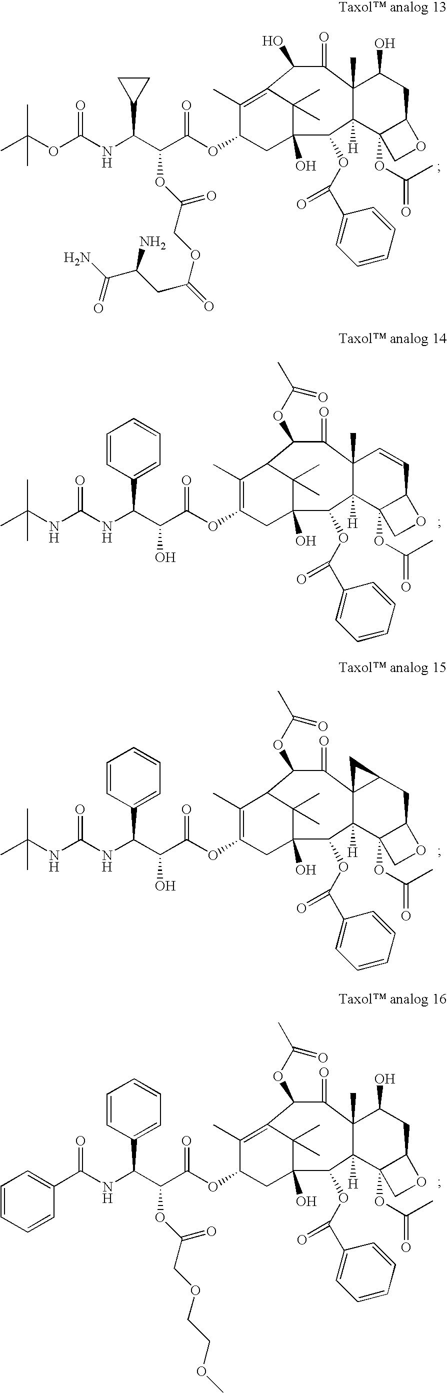

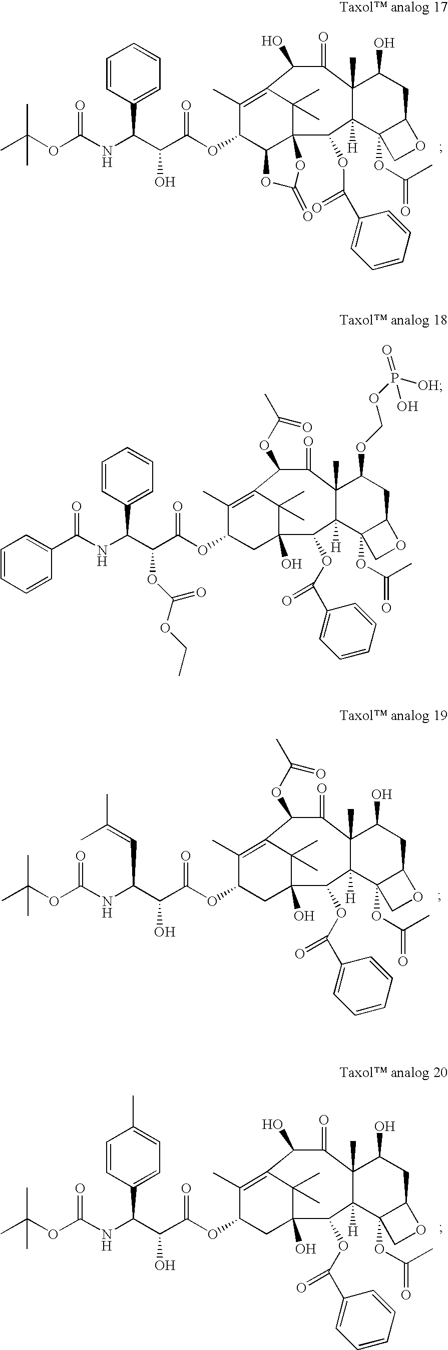

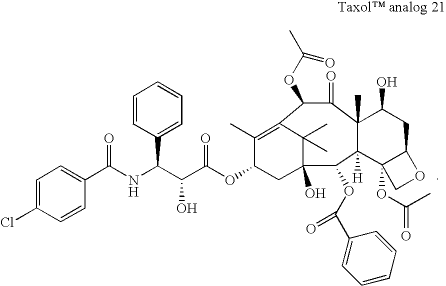

A Taxol™ analog can also be bonded to or be pendent from a pharmaceutically acceptable polymer, such as a polyacrylamide. One example of a polymer of this type is Taxol™ analog 22, below, which has the structure of a polymer comprising a taxol analog group pendent from the polymer backbone. The polymer is a terpolymer of the three monomer units shown. The term “Taxol™ analog”, as it is used herein, includes such polymers.

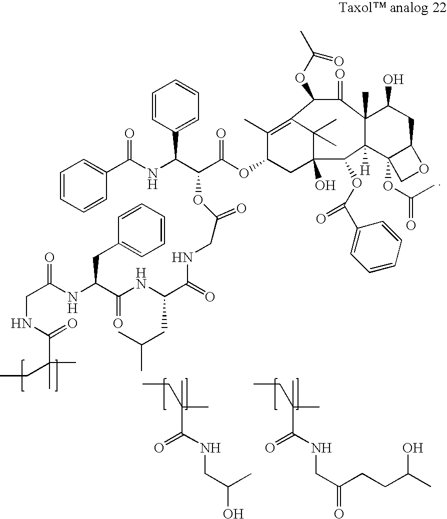

Other anti-cancer agents that can be employed in combination with bis(thio-hydrazide amides) described herein include Adriamycin, Dactinomycin, Bleomycin, Vinblastine, Cisplatin, acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cladribine; crisnatol mesylate; cyclophosphamide; cytarabine; dacarbazine; daunorubicin hydrochloride; decitabine; dexormaplatin; dezaguanine; dezaguanine mesylate; diaziquone; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other anti-cancer drugs that can be employed in combination with bis(thio-hydrazide amides) described herein include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorlns; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; 9-dioxamycin; diphenyl spiromustine; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; fornestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; ondansetron; ondansetron; oracin; oral cytokine inducer; ormaplatin; osaterone; oxaliplatin; oxaunomycin; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen-binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. Preferred anti-cancer drugs are 5-fluorouracil and leucovorin.

Chemotherapeutic agents that can be employed in combination with bis(thio-hydrazide amides) described herein include but are not limited to alkylating agents, antimetabolites, natural products, or hormones. Examples of alkylating agents useful for the treatment or prevention of T-cell malignancies in the methods and compositions of the invention include but are not limited to, nitrogen mustards (e.g., mechloroethamine, cyclophosphamide, chlorambucil, etc.), alkyl sulfonates (e.g., busulfan), nitrosoureas (e.g., carmustine, lomusitne, etc.), or triazenes (decarbazine, etc.). Examples of antimetabolites useful for the treatment or prevention of T-cell malignancies in the methods and compositions of the invention include but are not limited to folic acid analog (e.g., methotrexate), or pyrimidine analogs (e.g., Cytarabine), purine analogs (e.g., mercaptopurine, thioguanine, pentostatin). Examples of natural products useful for the treatment or prevention of T-cell malignancies in the methods and compositions of the invention include but are not limited to vinca alkaloids (e.g., vinblastin, vincristine), epipodophyllotoxins (e.g., etoposide), antibiotics (e.g., daunorubicin, doxorubicin, bleomycin), enzymes (e.g., L-asparaginase), or biological response modifiers (e.g., interferon alpha).

Examples of alkylating agents that can be employed in combination with bis(thio-hydrazide amides) described herein include but are not limited to, nitrogen mustards (e.g., mechloroethamine, cyclophosphamide, chlorambucil, melphalan, etc.), ethylenimine and methylmelamines (e.g., hexamethlymelamine, thiotepa), alkyl sulfonates (e.g., busulfan), nitrosoureas (e.g., carmustine, lomusitne, semustine, streptozocin, etc.), or triazenes (decarbazine, etc.). Examples of antimetabolites useful for the treatment or prevention of cancer in the methods and compositions of the invention include but are not limited to folic acid analog (e.g., methotrexate), or pyrimidine analogs (e.g., fluorouracil, floxouridine, Cytarabine), purine analogs (e.g., mercaptopurine, thioguanine, pentostatin). Examples of natural products useful for the treatment or prevention of cancer in the methods and compositions of the invention include but are not limited to vinca alkaloids (e.g., vinblastin, vincristine), epipodophyllotoxins (e.g., etoposide, teniposide), antibiotics (e.g., actinomycin D, daunorubicin, doxorubicin, bleomycin, plicamycin, mitomycin), enzymes (e.g., L-asparaginase), or biological response modifiers (e.g., interferon alpha). Examples of hormones and antagonists useful for the treatment or prevention of cancer in the methods and compositions of the invention include but are not limited to adrenocorticosteroids (e.g., prednisone), progestins (e.g., hydroxyprogesterone caproate, megestrol acetate, medroxyprogesterone acetate), estrogens (e.g., diethlystilbestrol, ethinyl estradiol), antiestrogen (e.g., tamoxifen), androgens (e.g., testosterone propionate, fluoxymesterone), antiandrogen (e.g., flutamide), gonadotropin releasing hormone analog (e.g., leuprolide). Other agents that can be used in the methods and compositions of the invention for the treatment or prevention of cancer include platinum coordination complexes (e.g., cisplatin, carboblatin), anthracenedione (e.g., mitoxantrone), substituted urea (e.g., hydroxyurea), methyl hydrazine derivative (e.g., procarbazine), adrenocortical suppressant (e.g., mitotane, aminoglutethimide).

Without wishing to be bound by theory, the bis(thio-hydrazide amides) are believed to be particularly effective when co-administered with anti-cancer agents which act by arresting cells in the G2-M phases due to stabilized microtubules, such as Taxol™, and Taxol™ analogs, as described above. Thus, the disclosed method preferably includes co-administered anti-cancer drugs which act by this mechanism. Other examples of anti-cancer agents which act by arresting cells in the G2-M phases due to stabilized microtubules include without limitation the following marketed drugs and drugs in development: Erbulozole (also known as R-55104), Dolastatin 10 (also known as DLS-10 and NSC-376128), Mivobulin isethionate (also known as CI-980), Vincristine, NSC-639829, Discodermolide (also known as NVP-XX-A-296), ABT-751 (Abbott, also known as E-7010), Altorhyrtins (such as Altorhyrtin A and Altorhyrtin C), Spongistatins (such as Spongistatin 1, Spongistatin 2, Spongistatin 3, Spongistatin 4, Spongistatin 5, Spongistatin 6, Spongistatin 7, Spongistatin 8, and Spongistatin 9), Cemadotin hydrochloride (also known as LU-103793 and NSC-D-669356), Epothilones (such as Epothilone A, Epothilone B, Epothilone C (also known as desoxyepothilone A or dEpoA), Epothilone D (also referred to as KOS-862, dEpoB, and desoxyepothilone B), Epothilone E, Epothilone F, Epothilone B N-oxide, Epothilone A N-oxide, 16-aza-epothilone B, 21-aminoepothilone B (also known as BMS-310705), 21-hydroxyepothilone D (also known as Desoxyepothilone F and dEpoF), 26-fluoroepothilone), Auristatin PE (also known as NSC-654663), Soblidotin (also known as TZT-1027), LS-4559-P (Pharmacia, also known as LS-4577), LS-4578 (Pharmacia, also known as LS-477-P), LS-4477 (Pharmacia), LS-4559 (Pharmacia), RPR-112378 (Aventis), Vincristine sulfate, DZ-3358 (Daiichi), FR-182877 (Fujisawa, also known as WS-9885B), GS-164 (Takeda), GS-198 (Takeda), KAR-2 (Hungarian Academy of Sciences), BSF-223651 (BASF, also known as ILX-651 and LU-22365 1), SAH-49960 (Lilly/Novartis), SDZ-268970 (Lilly/Novartis), AM-97 (Armad/Kyowa Hakko), AM-132 (Armad), AM-138 (Armad/Kyowa Hakko), IDN-5005 (Indena), Cryptophycin 52 (also known as LY-355703), AC-7739 (Ajinomoto, also known as AVE-8063A and CS-39.HCl), AC-7700 (Ajinomoto, also known as AVE-8062, AVE-8062A, CS-39-L-Ser.HCl, and RPR-258062A), Vitilevuamide, Tubulysin A, Canadensol, Centaureidin (also known as NSC-106969), T-138067 (Tularik, also known as T-67, TL-138067 and TI-138067), COBRA-1 (Parker Hughes Institute, also known as DDE-261 and WHI-261), H10 (Kansas State University), H16 (Kansas State University), Oncocidin A1 (also known as BTO-956 and DIME), DDE-313 (Parker Hughes Institute), Fijianolide B, Laulimalide, SPA-2 (Parker Hughes Institute), SPA-1 (Parker Hughes Institute, also known as SPIKET-P), 3-IAABU (Cytoskeleton/Mt. Sinai School of Medicine, also known as MF-569), Narcosine (also known as NSC-5366), Nascapine, D-24851 (Asta Medica), A-105972 (Abbott), Hemiasterlin, 3-BAABU (Cytoskeleton/Mt. Sinai School of Medicine, also known as MF-191), TMPN (Arizona State University), Vanadocene acetylacetonate, T-138026 (Tularik), Monsatrol, Inanocine (also known as NSC-698666), 3-IAABE (Cytoskeleton/Mt. Sinai School of Medicine), A-204197 (Abbott), T-607 (Tularik, also known as T-900607), RPR-115781 (Aventis), Eleutherobins (such as Desmethyleleutherobin, Desaetyleleutherobin, Isoeleutherobin A, and Z-Eleutherobin), Caribaeoside, Caribaeolin, Halichondrin B, D-64131 (Asta Medica), D-68144 (Asta Medica), Diazonamide A, A-293620 (Abbott), NPI-2350 (Nereus), Taccalonolide A, TUB-245 (Aventis), A-259754 (Abbott), Diozostatin, (−)-Phenylahistin (also known as NSCL-96F037), D-68838 (Asta Medica), D-68836 (Asta Medica), Myoseverin B, D-43411 (Zentaris, also known as D-81862), A-289099 (Abbott), A-318315 (Abbott), HTI-286 (also known as SPA-110, trifluoroacetate salt) (Wyeth), D-82317 (Zentaris), D-82318 (Zentaris), SC-12983 (NCI), Resverastatin phosphate sodium, BPR-0Y-007 (National Health Research Institutes), and SSR-250411 (Sanofi).

In addition, agents that modulate immune regulatory proteins (such as HSP90 inhibitors and HSP70 inducers) may also be particularly effective in combination with the compounds of this invention.

As used herein, a “subject” is a mammal, preferably a human, but can also be an animal in need of veterinary treatment, e.g., companion animals (e.g., dogs, cats, and the like), farm animals (e.g., cows, sheep, pigs, horses, and the like) and laboratory animals (e.g., rats, mice, guinea pigs, and the like).

As used herein, an “effective amount” is the quantity of compound in which a beneficial clinical outcome is achieved when the compound is administered to a subject. A “beneficial clinical outcome” includes reduction or inhibition of cell growth, a reduction in the severity of the symptoms associated with the cell growth (e.g., inhibition of restenosis, reduction of symptoms of psoriasis, reduction of pain associated with blocked coronary arteries, and the like). The precise amount of compound administered to a subject will depend on the mode of administration, the type and severity of the disease or condition and on the characteristics of the subject, such as general health, age, sex, body weight and tolerance to drugs. It will also depend on the degree, severity and type of cell proliferation, and the mode of administration. The skilled artisan will be able to determine appropriate dosages depending on these and other factors. For example, in some embodiments, a coating composition on a stent typically contains amounts of the bis(thio-hydrazide amides) in the range of between about 1 μg and about 100 mg, and may deliver that amount of drug over a time period ranging from several minutes to several weeks. When co-administered with other agents, e.g., when co-administered with an anti-cancer agent, an “effective amount” of the second agent will depend on the type of drug used. Suitable dosages are known for approved agents and can be adjusted by the skilled artisan according to the condition of the subject, the type of condition(s) being treated and the amount of a bis(thio-hydrazide amide) being used. In cases where no amount is expressly noted, an effective amount should be assumed.

The bis(thio-hydrazide amides) described herein can be administered to a subject by any conventional method of drug administration for treatment of non-cancerous proliferative disorders, for example, orally in capsules, suspensions or tablets or by parenteral administration. Parenteral administration can include, for example, systemic administration, such as by intramuscular, intravenous, subcutaneous, or intraperitoneal injection. The compounds can also be administered orally (e.g., dietary), topically, by inhalation (e.g., intrabronchial, intranasal, oral inhalation or intranasal drops), rectally. vaginally, and the like. In specific embodiments, oral, parenteral, or local administration are preferred modes of administration for treatment of non-cancerous proliferative disorders.

The bis(thio-hydrazide amides) described herein can be administered to the subject in conjunction with an acceptable pharmaceutical carrier or diluent as part of a pharmaceutical composition for treatment of non-cancerous proliferative disorders. Formulation of the compound to be administered will vary according to the route of administration selected (e.g., solution, emulsion, capsule, and the like). Suitable pharmaceutically acceptable carriers may contain inert ingredients which do not unduly inhibit the biological activity of the compounds. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in Remington's Pharmaceutical Sciences, ibid. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al., “Controlled Release of Biological Active Agents”, John Wiley and Sons, 1986).

The bis(thio-hydrazide amides) described herein can be administered to a subject for treatment of proliferative disorders including cancer by contacting the subject with the medical devices described above.

The amount of the bis(thio-hydrazide amide) or composition comprising the bis(thio-hydrazide amide) which will be effective in the prevention, treatment, management, or amelioration of a proliferative disorder or one or more symptoms thereof will vary with the nature and severity of the disease or condition, and the route by which the active ingredient is administered. The frequency and dosage will also vary according to factors specific for each patient depending on the specific therapy (e.g., therapeutic or prophylactic agents) administered, the severity of the disorder, disease, or condition, the route of administration, as well as age, body, weight, response, and the past medical history of the patient. Effective doses may be extrapolated from dose-response curves derived from in vitro or animal model test systems. Suitable regiments can be selected by one skilled in the art by considering such factors and by following, for example, dosages reported in the literature and recommended in the Physician 's Desk Reference (57th ed., 2003).

Exemplary doses of the bis(thio-hydrazide amide) include milligram or microgram amounts of the compound per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). Typically, a larger dose per kilogram of body weight is needed when the bis(thio-hydrazide amide) is delivered from a device that is remote from the treatment site than when the device is located at the treatment site.

In general, the recommended daily dose range of a compound of the invention for the conditions described herein lie within the range of from about 0.01 mg to about 3000 mg per day, given as a single once-a-day dose or, as divided doses throughout a day, or preferably, continuously via a medical device. Specifically, a daily dose range should be from about 5 mg to about 500 mg per day, more specifically, between about 10 mg and about 200 mg per day. In managing the patient, the therapy should be initiated at a lower dose, perhaps about 1 mg to about 25 mg, and increased if necessary up to about 200 mg to about 1000 mg per day as either a single dose or divided doses, depending on the patient's global response. In one embodiment, the dose is delivered from a medical device that is located at a treatment site. In this embodiment, a lower dose is typically effective (e.g., about 0.01 mg to about 25 mg per day). It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

Different therapeutically effective amounts may be applicable for different proliferative disorders, as will be readily known by those of ordinary skill in the art. Similarly, amounts sufficient to prevent, manage, treat or ameliorate such proliferative disorder, but insufficient to cause, or sufficient to reduce, adverse effects associated with the compounds of the invention are also encompassed by the above described dosage amounts and dose frequency schedules. Further, when a patient is administered multiple dosages of a compound of the invention, not all of the dosages need be the same. For example, the dosage administered to the patient may be increased to improve the prophylactic or therapeutic effect of the compound or it may be decreased to reduce one or more side effects that a particular patient is experiencing.

In a specific embodiment, the dosage of the composition of the invention or a compound of the invention administered to prevent, treat, manage, or ameliorate a cell proliferative disorder or one or more symptoms thereof in a patient is 150 μg/kg, preferably 250 μg/kg, 500 μg/kg, 1 mg/kg, 5 mg/kg, 10 mg/kg, 25 mg/kg, 50 mg/kg, 75 mg/kg, 100 mg/kg, 125 mg/kg, 150 mg/kg, or 200 mg/kg or more of a patient's body weight. In another embodiment, the dosage of the composition of the invention or a compound of the invention administered to prevent, treat, manage, or ameliorate a proliferative disorder or one or more symptoms thereof in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7m g, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

The dosages of prophylactic or therapeutic agents other than compounds of the invention, which have been or are currently being used to prevent, treat, manage, or ameliorate a proliferative disorder or one or more symptoms thereof can be used in the combination therapies of the invention. Preferably, dosages lower than those which have been or are currently being used to prevent, treat, manage, or ameliorate a proliferative disorder or one or more symptoms thereof are used in the combination therapies of the invention. The recommended dosages of agents currently used for the prevention, treatment, management, or amelioration of a proliferative disorder or one or more symptoms thereof can obtained from any reference in the art including, but not limited to, Hardman et al., eds., 1996, Goodman & Gilman's The Pharmacological Basis Of Basis Of Therapeutics 9th Ed, Mc-Graw-Hill, New York; Physician's Desk Reference (PDR) 57^{th} Ed., 2003, Medical Economics Co., Inc., Montvale, N.J., which are incorporated herein by reference in its entirety.

In various embodiments, the therapies (e.g., prophylactic or therapeutic agents) are administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In preferred embodiments, two or more therapies (e.g., prophylactic or therapeutic agents) are administered within the same patent visit. In another preferred embodiment, two or more therapies are administered from one medical device.

In certain embodiments, one or more compounds of the invention and one or more other the therapies (e.g., prophylactic or therapeutic agents) are cyclically administered. Cycling therapy involves the administration of a first therapy (e.g., a first prophylactic or therapeutic agents) for a period of time, followed by the administration of a second therapy (e.g., a second prophylactic or therapeutic agents) for a period of time, followed by the administration of a third therapy (e.g., a third prophylactic or therapeutic agents) for a period of time and so forth, and repeating this sequential administration, i.e., the cycle in order to reduce the development of resistance to one of the agents, to avoid or reduce the side effects of one of the agents, and/or to improve the efficacy of the treatment.

In certain embodiments, administration of the same compound of the invention may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In a specific embodiment, the invention provides a method of preventing, treating, managing, or ameliorating a proliferative disorder or one or more symptoms thereof, said methods comprising administering to a subject in need thereof a dose of at least 150 μg/kg, preferably at least 250 μg/kg, at least 500 μg/kg, at least 1 mg/kg, at least 5 mg/kg, at least 10 mg/kg, at least 25 mg/kg, at least 50 mg/kg, at least 75 mg/kg, at least 100 mg/kg, at least 125 mg/kg, at least 150 mg/kg, or at least 200 mg/kg or more of one or more compounds of the invention once every day, preferably, once every 2 days, once every 3 days, once every 4 days, once every 5 days, once every 6 days, once every 7 days, once every 8 days, once every 10 days, once every two weeks, once every three weeks, or once a month.

In one embodiment, a compound of the invention is delivered locally to a treatment site by contacting the treatment site with a medical device that include the compound in a reservoir, coating composition or controlled release polymer matrix. For example, the compound may be included in a coating composition or reservoir in a stent. For purposes of local delivery from a stent which has a reservoir, coating composition or controlled release polymer matrix that comprises a compound of the invention, the daily dose that a patient will receive of the compound of the invention depends on the length of the stent. For example, a coronary stent may contain a drug in an amount ranging from 0.0001 mg to 1000 mg, preferably 0.001 mg to 500 mg, more preferably 0.001 mg to 100 mg. This dose may be delivered over a time period ranging from several hours to several weeks.

Also included in the present invention are pharmaceutically acceptable salts of the bis(thio-hydrazide amides) described herein. These bis(thio-hydrazide amides) can have one or more sufficiently acidic protons that can react with a suitable organic or inorganic base to form a base addition salt. Base addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

For example, pharmaceutically acceptable salts of the bis(thio-hydrazide amides) (e.g., those represented by Structural Formulas I-V or Compounds 1-18) are those formed by the reaction of the bis(thio-hydrazide amide) with one equivalent of a suitable base to form a monovalent salt (i.e., the compound has single negative charge that is balanced by a pharmaceutically acceptable counter cation, e.g., a monovalent cation) or with two equivalents of a suitable base to form a divalent salt (e.g., the compound has a two-electron negative charge that is balanced by two pharmaceutically acceptable counter cations, e.g., two pharmaceutically acceptable monovalent cations or a single pharmaceutically acceptable divalent cation). Divalent salts of the bis(thio-hydrazide amides) are preferred. “Pharmaceutically acceptable” means that the cation is suitable for administration to a subject. Examples include Li⁺, Na⁺, K⁺, Mg²⁺, Ca²⁺]and NR₄⁺, wherein each R is independently hydrogen, an optionally substituted aliphatic group (e.g., a hydroxyalkyl group, aminoalkyl group or ammoniumalkyl group) or optionally substituted aryl group, or two R groups, taken together, form an optionally substituted non-aromatic heterocyclic ring optionally fused to an aromatic ring. Generally, the pharmaceutically acceptable cation is Li⁺, Na⁺, K⁺, NH₃(C₂H₅OH)⁺or N(CH₃)₃(C₂H₅OH)⁺, and more typically, the salt is a disodium or dipotassium salt, preferably the disodium salt.

Bis(thio-hydrazide amides) with a sufficiently basic group, such as an amine can react with an organic or inorganic acid to form an acid addition salt. Acids commonly employed to form acid addition salts from compounds with basic groups are inorganic acids such as hydrochloric acid, hydrobromic acid, hydroiodic acid, sulfuric acid, phosphoric acid, and the like, and organic acids such as p-toluenesulfonic acid, methanesulfonic acid, oxalic acid, p-bromophenyl-sulfonic acid, carbonic acid, succinic acid, citric acid, benzoic acid, acetic acid, and the like. Examples of such salts include the sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caproate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, sulfonate, xylenesulfonate, phenylacetate, phenylpropionate, phenylbutyrate, citrate, lactate, gamma-hydroxybutyrate, glycolate, tartrate, methanesulfonate, propanesulfonate, naphthalene-1-sulfonate, naphthalene-2-sulfonate, mandelate, and the like.

Salts of the bis(thio-hydrazide amide) compounds described herein can be prepared according to methods described in a copending and co-owned Patent Application Ser. No. 60/582,596, filed Jun. 23, 2004. The neutral compounds can be prepared according to methods described in U.S. Publication Nos. 2003/0045518 and 2003/0119914, both entitled “Synthesis of Taxol Enhancers” and also according to methods described in the co-pending and co-owned U.S. application Ser. No. 10/758,589, entitled “Treatment for Cancers”, filed Jan. 15, 2004. For avoidance of confusion, the treatment methods described and claimed herein specifically exclude the treatment methods described in the patent filings mentioned in this paragraph. The entire teachings of each document referred to in this application is expressly incorporated herein by reference.

It will also be understood that certain compounds of the invention may be obtained as different stereoisomers (e.g., diastereomers and enantiomers) and that the invention includes all isomeric forms and racemic mixtures of the disclosed compounds and methods of treating a subject with both pure isomers and mixtures thereof, including racemic mixtures. Stereoisomers can be separated and isolated using any suitable method, such as chromatography.

### EXEMPLIFICATION

The present invention is illustrated by the following example, which are not intended to be limiting in any way. The structures of compounds (1)-(18) in the following are depicted in the Detailed Description above.

### Example 1

### Multi-Drug Resistant Specific Anti-Cancer Activity Demonstrated In Vitro

The in vitro activity of the compounds was assessed in a selected set of human cancer cell lines. Three pairs of tumor cell lines (non-resistant/resistant) were used to identify novel potent antitumor compounds which are capable of overcoming multi-drug resistance.

HL-60, a model of myeloid leukemia, was obtained from ATCC (ATCC CCL-240); and HL60/TX1000 was isolated in vitro by subculturing HL-60 in progressively higher concentration of Taxol™. HL-60/TX1000 cells over-express mdr-1 mRNA and p-glycoprotein (PCP), as determined by western blot and immunofluorescence labeling with antiPGP antibodies. The cells are cross-resistant to Taxol™, Vincristine, Adriamycin, Etoposide and Doxorubicin.

MES—SA, a model of uterine sarcoma, is sensitive to a number of chemotherapeutic agents, including Doxorubicin, Dactinomycin, Mitomycin C, Taxol™ and Bleomycin, but resistant to Vinblastine and Cisplatin. MES—SA/DX5 was established in the presence of increasing concentrations of Doxorubicin. The cells express high levels of mdr-1 mRNA and p-glycoprotein and exhibit cross resistance to more than fifteen chemotherapeutic agents including Taxol™, Etoposide, Mitomycin C, Colchicine, Vinblastine, Dactinomycin, 5-Fluorouracil, Methotrexate and others. Both MES—SA and MES—SA/Dx5 were purchased from ATCC (ATCC CRL-1976 and ATCC CRL-1977, respectively).

Bowes is a melanoma cell line; and Bowes/OV2 is a Vincristine resistant Bowes melanoma cell line.

The cell lines were maintained in RPMI1640 (GIBCO) supplemented with 10% FCS, 100 units/ml penicillin, 100 ug/ml streptomycin, and 2 mM L-glutamine. The cells were split every third day and diluted to a concentration of 2×10⁵ cells/ml one day before experiment. All experiments were performed on exponentially growing cell culture. Cell densities were 2.5×10⁴ cells/ml in all experiment except special.

A stock solution of Compound (1), Taxol™ (positive control) and Vincristine (positive control) were prepared by dissolving the compound at a concentration of 1 mM in 100% DMSO. Final concentrations were obtained by diluting the stock solution directly into the tissue culture medium. Cells were incubated with varying concentrations of compounds for 72 hours and the IC₅₀ was determined by MTS (i.e. 3-(4.5-dimethylthiazol-2-yl)-2.5-diphenyl tetrazolium bromide) assay. The IC₅₀ is the concentration of compound required to inhibit 50% tumor cell growth. The results are shown in Table 1.

TABLE 1
Inhibition of Growth of Multi-Drug Resistant Tumor Cell Lines
by Anti-Cancer Agents and Compound (1)

| | IC₅₀ (uM) | | | | | |
|---|---|---|---|---|---|---|
| | MES- | MES- | HL- | HL- | | Bowes/ |
| | SA | SA/DX5 | 60 | 60/TX1000 | Bowes | OV2 |
| Taxol ™ | 0.005 | 5 | 0.002 | 5 | 0.005 | 5 |
| Vincristine | 0.004 | 5 | 0.002 | 5 | 0.002 | 5 |
| Compound | 0.05 | 0.005 | 0.4 | 0.05 | 0.2 | 0.01 |
| (1) | | | | | | |

As can be seen from the data in Table 1, Taxol™ and Vincristine demonstrated significantly high anti-cancer activity (IC₅₀: 0.002-0.005 uM) agaist normal cancer cell lines (MES—SA, HL-60, Bowes). However, these anti-cancer drugs were significantly less effective (IC₅₀: 5 uM) against the MDR cell lines (MES—SA/DX5, HL-60/TX1000, Bowes/OV2). On the other hand, Compound (1) surprisingly showed higher anti-cancer activity against all three MDR cell lines. The specificity were 10 (=0.05/0.005), 8 (=0.4/0.05), and 20 (=0.2/0.01) against MES—SA/DX5, HL60/TX1000, and Bowes/OV2, respectively.

### Example 2

### Compounds (2)-(18) Demonstrate High Anti-Cancer Activity Against Multi-Drug Resistant MES—SA/DX5 In Vitro

The protocol described in Example 1 was used to test Compounds (2)-(18) for investigating inhibitory activity of cancer cell growth of MES—SA/DX5, which is a MDR uterine sarcoma cell line. The results are shown in Table 2, below.

As can be seen from the data in Table 2, Compounds (2)-(18) demonstrated significant anti-cancer activity (IC₅₀: 0.05-0.005 uM) against the multi-drug resistant (MDR) cell line MES—SA/DX5, while Taxol™ showed very week anti-cancer activity (IC₅₀: 5 uM) against the same MDR cell line.

TABLE 2
Inhibition of Growth of the Multi-Drug Resistant Tumor Cell Line MES-
SA/DX5 by Compounds (2)-(18).

| | IC₅₀ (uM) |
|---|---|
| Compound | MES/DX5 |
| Taxol ™ | 5 |
| 2 | 0.005 |
| 3 | 0.05 |
| 4 | 0.005 |
| 5 | 0.05 |
| 6 | 0.005 |
| 7 | 0.01 |
| 8 | 0.005 |
| 9 | 0.005 |
| 10 | 0.005 |
| 11 | 0.005 |
| 12 | 0.005 |
| 13 | 0.05 |
| 14 | 0.01 |
| 15 | 0.005 |
| 16 | 0.05 |
| 17 | 0.005 |
| 18 | 0.01 |

### Example 3

### Compound (16) Demonstrates Anti-Cancer Activity Against Multi-Drug Resistant Human Uterine Sarcoma MES/SA-DX5 Tumors in Nude Mice

A supplemented media was prepared from 50% DMEM/Dulbecco Modified Eagle Medium (High Glucose), 50% RPMI 1640, 10% FBS/Fetal Bovine Serum (Hybridoma Tested; Sterile Filtered), 1% L-Glutamine, 1% Penicillin-Streptomycin, 1% MEM Sodium Pyruvate and 1% MEM Non-Essential Amino Acids. FBS was obtained from Sigmna Chemical Co. and other ingredients were obtained from Invitrogen Life Technologies, USA). The supplemental media was warmed to 37° C. and 50 ml of media was added to a 175 cm² tissue culture flask.

The cells used in the assay were multi-drug resistant MES—SA/DX-5 Human Uterine Sarcoma cells from the American Type Culture Collection. 1 vial of MES—SA/DX-5 cells from the liquid nitrogen frozen cell stock was removed. The frozen vial of cells was immediately placed into a 37° C. water bath and gently swirled until thawed. The freeze-vial was wiped with 70% ethanol and cells were immediately pipetted into the 175 cm² tissue culture flask containing supplemented media. The cells were incubated overnight and the media was removed and replaced with fresh supplemented media the next day. The flask was incubated until the cells became about 90% confluent. This took anywhere from 5-7 days.

The flask was washed with 10 ml of sterile room temperature phosphate buffered saline (PBS). The cells were trypsinized by adding 5 ml of warmed Trypsin-EDTA (Invitrogen) to the flask of cells. The cells were then incubated for 2-3 minutes at 37° C. until cells begun to detach from the surface of the flask. An equal volume of supplemented media (5 ml) was added to the flask. All the cells were collected into 50 ml tube, and centrifuged at 1000 RPM for 5 minutes at 20° C. The supernatant was aspirated and the cell pellet was resuspended in 10 ml of supplemented media and the cells were counted. 1-3 million cells/flask were seeded into 5-7 tissue culture flasks (175 cm²). Each flask contained 50 ml of supplemented media. The flasks were incubated until about 90% confluent. The passaging of the cells was repeated until enough cells had been grown for tumor implantation.

The above procedure for trypsinizing and centrifuging the cells were followed. The supernatant was aspirated and the cell pellet was resuspended in 10 ml of sterile PBS and the cells were counted. The cells were centrifuged and then resuspended with appropriate volume of sterile PBS for injection of correct number of cells needed for tumor implantation. 100 million cells were suspended with 2.0 ml of sterile PBS to a final concentration of 50 million cells/ml in order to inject 5 million cells in 0.1 ml/mouse.

Five million MES—SA/DX5 cells were injected subcutaneously into the flan (lateral side) of female CB.17/SCID mice (Age 6-7 wks). These mice were obtained from Taconic, Germantown, N.Y. (Nomenclature: C.B-Igh-1^{b}IcrTac-Prkdc^{scid}) CB.17/SCID (FOX CHASE SCID) and are homozygous for the autosomal recessive scid (severe combined immunodeficient) gene and lack both T and B cells due to a defect in V(D)J recombination. Therefore, they easily accept foreign tissue transplants. These tumors were allowed to grow until they reached a size of about 200-300 mm³ before they were excised and prepared as a single cell suspension. These cells were then seeded into tissue culture flasks. The cells went through two passages in vitro before the tumor cells were collected.

Mice (CD-1 nu/nu) were obtained from Charles River Laboratories: nomenclature: Crl:CD-1-nuBR, Age: 6-8 weeks. The mice were allowed to acclimate for 1 week prior to their being used in an experimental procedure.

Implantation of the MES—SA/DX5 tumor cell suspension took place in the lateral flank of the female CD-1 nu/nu mouse. Five million tumor cells in 0.1 mL of PBS were injected using a 27G (½ inch) needle. MES—SA/DX5 tumors developed after 2-3 weeks after implantation.

Compound stock solutions were prepared by dissolving the compound in cell-culture-grade DMSO (dimethyl sulfoxide) at the desired concentration. This stock solution in DMSO was sonicated in an ultrasonic water bath until all the powder dissolved.

The Formulation Solvent was prepared as follows: 20% of Cremophore RH40 (Polyoxyl 40 Hydrogenated Castor Oil obtained from BASF corp.) in water was prepared by first heating 100% Cremophore RH40 in a water bath at 50-60° C. until it liquefied and became clear. 10 ml of the 100% Cremophore RH40 aliquoted into a conical centrifuge tube containing 40 ml of sterile water (1:5 dilution of Cremophore RH40). The 20% Cremophore RH40 solution was reheated until it became clear again, and mixed by inverting the tube several times. This 20% Cremophore RH40 solution was stored at room temperature, and was kept for up to 3 months.

Preparation of Dosing Solution for Compound Administration: The compound stock solution was diluted 1:10 with 20% Cremophore RH40: 1) 2.0 ml of 10 mg/ml dosing solution of Compound (16) was prepared by diluting 100 mg/ml Compound Stock solution with 1.8 ml of 20% Cremophore RH40 water solution. The final formulation for the dosing solution was 10% DMSO, 18% Cremophore RH40 and 72% water.

The Dosing Solution (Dosing Volume: 0.01 ml/gram=10 ml/kg) was injected intravenously into the mice bearing MES—SA/DX-5 human sarcoma tumor.

PROTOCOL

| Group | Compound | (Dose) |
|---|---|---|
| 1 | Vehicle Only | |
| 2 | Compound (16) | (15 mg/kg) |

Dosing Schedule : 3 times a week (Monday, Wednesday, Friday) for 3 weeks 5 mice were used for each group

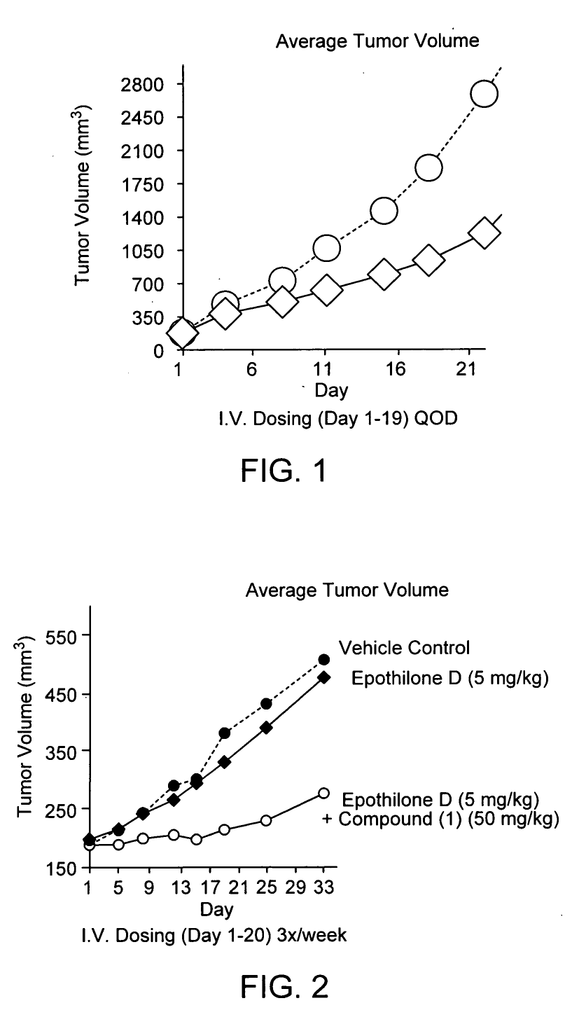

FIG. 1 shows the effects of Compound (16) on inhibiting tumor growth of MES/SA-DX5. As can be seen from FIG. 1, Compound (16) significantly inhibits the tumor growth compared to vehical treated mice. In addition, the mice showed no obvious toxicity such as body weight suppression and behavior changes.

### Example 4

### Combination Treatment of Compound (1) and Epothilone Demonstrated Anti-Tumor Activity Against Human Breast Carcinoma MDA-435 in Nude Mice

A supplemented media was prepared from 50% DMEM/Dulbecco Modified Eagle Medium (High Glucose), 50% RPMI 1640, 10% FBS/Fetal Bovine Serum (Hybridoma Tested; Sterile Filtered), 1% L-Glutamine, 1% Penicillin-Streptomycin, 1% MEM Sodium Pyruvate and 1% MEM Non-Essential Amino Acids. FBS was obtained from Sigma Chemical Co. and other ingredients were obtained from Invitrogen Life Technologies, USA). The supplemental media was warmed to 37° C and 50 ml of media was added to a 175 cm² tissue culture flask.

The cells used in the assay were MDA-435 Human Breast Carcinoma from the American Type Culture Collection. 1 vial of MDA-435 cells from the liquid nitrogen frozen cell stock was removed. The frozen vial of cells was immediately placed into a 37° C. water bath and gently swirled until thawed. The freeze-vial was wiped with 70% ethanol and cells were immediately pipetted into the 175 cm² tissue culture flask containing supplemented media. The cells were incubated overnight and the media was removed and replaced with fresh supplemented media the next day. The flask was incubated until flask became about 90% confluent. This took anywhere from 5-7 days.

The flask was washed with 10 ml of sterile room temperature phosphate buffered saline (PBS). The cells were trypsinized by adding 5 ml of warmed Trypsin-EDTA (Invitrogen) to the flask of cells. The cells were then incubated for 2-3 minutes at 37° C. until cells begun to detach from the surface of the flask. An equal volume of supplemented media (5 ml) was added to the flask. All the cells were collected into 50 ml tube, and centrifuged at 1000 RPM for 5 minutes at 20° C. The supernatant was aspirated and the cell pellet was resuspended in 10 ml of supplemented media and the cells were counted. 1-3 million cells/flask were seeded into 5-7 tissue culture flasks (175 cm²). Each flask contained 50 ml of supplemented media. The flasks were incubated until about 90% confluent. The passaging of the cells was repeated until enough cells have been grown for tumor implantation.

The above procedure for trypsinizing and centrifuging the cells were followed. The supernatant was aspirated and the cell pellet was resuspended in 10 ml of sterile PBS and the cells were counted. The cells were centrifuged and then resuspended with appropriate volume of sterile PBS for injection of correct number of cells needed for tumor implantation. In the case of MDA-435, 100 million cells were suspended with 2.0 ml of sterile PBS to a final concentration of 50 million cells/ml in order to inject 5 million cells in 0.1 ml/mouse.

Mice (CD-1 nu/nu) were obtained from Charles River Laboratories: nomenclature: Crl:CD-1-nuBR, Age: 6-8 weeks. The mice were allowed to acclimate for 1 week prior to their being used in an experimental procedure.

Implantation of the MDA-435 tumor cell suspension took place into the corpus adiposum of the female CD-1 nu/nu mouse. This fat body is located in the ventral abdominal viscera of the mouse. Tumor cells were implanted subsutaneously into the fat body located in the right quadrant of the abdomen at the juncture of the os coxae (pelvic bone) and the os femoris (femur). 5 million MDA-435 cells in 0.1 ml of sterile PBS were injected using 27 G (½ inch) needle. MDA-435 tumors developed 2-3 weeks after implantation.

Compound stock solutions were prepared by dissolving the compound in cell-culture-grade DMSO (dimethyl sulfoxide) at the desired concentration. This stock solution in DMSO was sonicated in an ultrasonic water bath until all the powder dissolved.

The Formulation Solvent was prepared as follows: 20% of Cremophore RH40 (Polyoxyl 40 Hydrogenated Castor Oil obtained from BASF corp.) in water was prepared by first heating 100% Cremophore RH40 in a water bath at 50-60° C. until it liquefied and became clear. 10 ml of the 100% Cremophore RH40 aliquoted into a conical centrifuge tube containing 40 ml of sterile water (1:5 dilution of Cremophore RH40). The 20% Cremophore RH40 solution was reheated until it became clear again, and mixed by inverting the tube several times. This 20% Cremophore RH40 solution was stored at room temperature, and was kept for up to 3 months.

Preparation of Dosing Solution for Compound Administration: The compound stock solution was diluted 1:10 with 20% Cremophore RH40: 1) 2.0 ml of 10 mg/ml dosing solution of Compound (1) was prepared by diluting 100 mg/ml Compound Stock solution with 1.8 ml of 20% Cremophore RH40 water solution; and 2) a dosoing solution comprising 2.0 ml of 1 mg/ml of Epothilone D and 5 mg/ml of Compound (1) was obtained by mixing 0.1 ml of Compound (1) DMSO stock solution (50 mg/ml) and 0.1 ml of Epothilone D DMSO stock solution (10 mg/ml) and diluting with 1.8 ml of 20% Cremophore RH40 water solution. The final formulation for the dosing solution was 10% DMSO, 18% Cremophore RH40 and 72% water.

The Dosing Solution (Dosing Volume: 0.01 ml/gram=10 ml/kg) was injected intravenously into the mice bearing MDA-435 human breast tumor.

PROTOCOL

| Group | Compounds | (Dose) |
|---|---|---|
| 1 | Vehicle Only | |
| 2 | Epothilone D | (5 mg/kg) |
| 3 | Epothilone D | (5 mg/kg) + Compound (1) (50 mg/kg) |

Dosing Schedule : 3 times a week (Monday, Wednesday, Friday) for 3 weeks. 5 mice were used for each group

FIG. 2 shows the effects of Compound (1) on enhancing anti-tumor activity of Epothilone D. As can be seen from FIG. 2, Compound (1) significantly enhanced anti-tumor activity of Epothilone D on human breast tumor MDA-435 in nude mice.

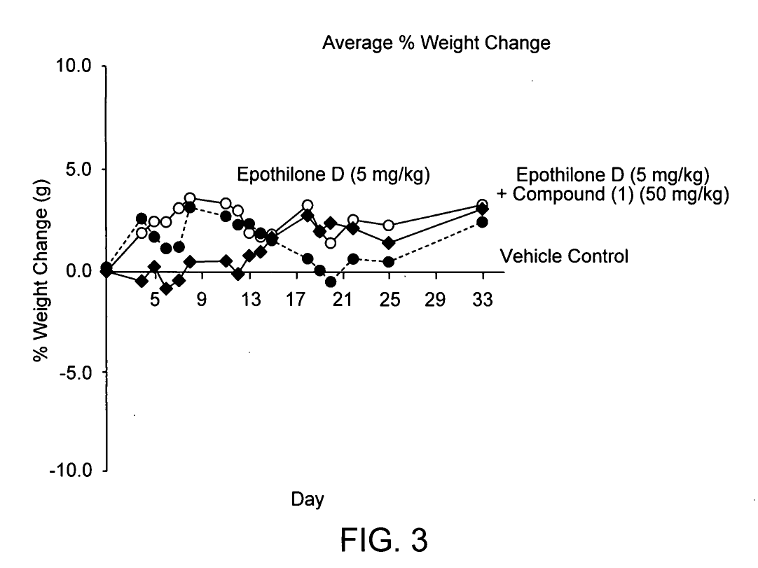

FIG. 3 shows the effects of treatment of Epothilone D and the combination of Compound (1) and Epothilone D on the body weight of nude mice bearing MDA-435 human breast tumor. As can be seen from FIGS. 2 and 3, Compound (1) enhanced anti-tumor activity of Epothilone D without increasing toxicity.

### Example 5

### Compound(l) Has Anti-leukemia Activity in vitro

The in vitro activity of the compounds was determined in a selected set of human leukemia cell lines. CEM (T-cell leukemia), Jurkat (T-cell leukemia), K562 (chronic myelocyte), THP-1 (monocyte), SB (B-cell leukemia), U937 (lymphoma) were purchased from ATCC. H2 leukemia cell line was a gift from Harvard Medical School. The cell lines were maintained in RPMI1640(GIBCO) supplemented with 10% FCS, 100 units/ml penicillin, 100 ug/ml streptomycin, and 2 mM L-glutamine. The cells were split every third day and diluted to a concentration of 2×105 cells/mL one day before experiment. All experiments were performed on exponentially growing cell culture. Cell densities were 2.5×104 cells/mL in all experiments.

Compound (1) was prepared by dissolving the compound at a concentration of 10 mM in 100% DMSO. Final concentrations 10, 1, 0.1, 0.01 and 0.001 M were obtained by diluting the stock solution directly into the tissue culture medium. Cells were incubated with varying concentrations of compounds for 72 hours and the IC50 was determined by MTS (i.e. 3-(4.5.-dimethylthiazol-2-yl)-2.5-diphenyl tetrazolium bromide) assay. IC50 is the concentration of compound required to inhibit 50% tumor cell growth. Table 3 shows the in vitro IC50 (μM) cytotoxicity results of Compound (1) versus vincristin and Taxol™.

TABLE 3
In vitro Cytotoxicity (IC50, μM) of Compound (1)
versus Vincristin and Taxol ™

| Cell | | | Compound | | |
|---|---|---|---|---|---|
| Line | Species | Cell type | (1) | Vincristin | Taxol ™ |
| 39SK | Human | normal | >10 | 1 | 1 |
| | | fibroblast | | | |
| Jurkat | Human | T cell leukemia | 0.005 | 0.001 | 0.001 |
| CEM | Human | T cell leukemia | 0.01 | 0.005 | 0.01 |
| K-562 | Human | chronic | 0.05 | 0.005 | 0.005 |
| | | myelocyte | | | |
| THP-1 | Human | monocyte | 0.01 | 0.005 | 0.005 |
| U937 | Human | lymphoma | 0.05 | 0.005 | 0.005 |
| SB | Human | B cell leukemia | 0.005 | 0.001 | 0.001 |
| H2 | Human | leukemia | 0.005 | 0.005 | 0.005 |

### Example 6

### Compound (1) Inhibits Human T-Cell Leukemia Growth (CEM Cell Line)

Human T-cell leukemia cell line, CEM, was obtained from American Type Culture Collection. Eight-week old female SCID mice were purchased from Charles Rive Laboratories (Wilmington, Mass.). FITC conjugated anti-human HLA-A,B,C was obtained from BD ParMingen (Cat # 32294X). ACK lysing buffer was obtained from BioWhittaker.

CEM cells (1×10⁶ cells in 100 μl saline) were implanted intravenously into female SCID mice through the tail vein. Vehicle and Compound (1) (25 mg/kg) were administrated intraperitoneally twice a day and total for 3 weeks. After three weeks treatment, blood was taken from mouse retro-orbital sinus at day 33. Red blood cells were partially lyzed with ACK lysing buffer. The cells were stained with FITC conjugated anti-human HLA-A,B,C antibody for one hour at 4° C. FACS analysis was performed to quantitate the amount of CEM cells in the blood. White blood cells were gated for FACS analysis. The results showed that about 37.7%, 4.6% and 1.07% of CEM cells were detected in the white blood cells from vehicle treated, Compound (1) treated, and untreated group respectively (Table 4).

TABLE 4
Summary of CEM cell quantitation at day 33

| | % circulating | |
|---|---|---|
| Treatment | leukemia cells | % relative to vehicle |
| Vehicle (n = 5) | 37.7 | 100 |
| Compound (1) (n = 5) | 4.6 | 12.2 |
| Untreated mice (n = 2) | 1.07 | 2.8 |

While this invention has been particularly shown and described with references to preferred embodiments thereof, it will be understood by those skilled in the art that various changes in form and details may be made therein without departing from the scope of the invention encompassed by the appended claims.

1. A method of treating a non-cancerous proliferative disorder in a subject, comprising administering to the subject an effective amount of a compound represented by the following Structural Formula: 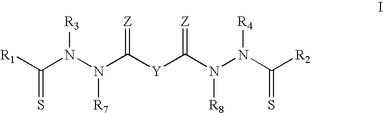
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S.

2. The method of claim 1, wherein the disorder is smooth muscle cell proliferation, systemic sclerosis, cirrhosis of the liver, adult respiratory distress syndrome, idiopathic cardiomyopathy, lupus erythematosus, retinopathy, cardiac hyperplasia, benign prostatic hyperplasia, ovarian cysts, pulmonary fibrosis, endometriosis, fibromatosis, harmatomas, lymphangiomatosis, sarcoidosis, desmoid tumors, intimal smooth muscle cell hyperplasia, restenosis, vascular occlusion, hyperplasia in the bile duct, hyperplasia in the bronchial airways, hyperplasia in the kidneys of patients with renal interstitial fibrosis, psoriasis, Reiter's syndrome, pityriasis rubra pilaris, a hyperproliferative disorder of keratinization, or scleroderma.

3. The method of claim 1, wherein the disorder is a proliferative vascular disorder.

4. The method of claim 1, wherein the disorder is a proliferative skin disorder.

5. The method of claim 1, wherein the disorder is a mechanically-mediated injury.

6. The method of claim 1, wherein the compound is administered to a treatment site in or on the subject.

7. The method of claim 4, wherein the compound is administered by application of a solution, cream, ointment or gel comprising the compound to the skin of the subject.

8. The method of claim 1, wherein the compound is administered to the treatment site in or on the subject by contacting the subject with a medical device which comprises the compound within a reservoir, coating composition or controlled release polymer matrix.

9. The method of claim 8, wherein the medical device is a transdermal patch that comprises a reservoir or a controlled release polymer matrix comprising the compound.

10. The method of claim 8, wherein the medical device is an osmotic pump.

11. The method of claim 8, wherein the treatment site is contacted with the medical device.

12. The method of claim 11, further comprising surgically inserting the medical device into the subject.

13. The method of claim 12 wherein the medical device is coated with a composition that comprises the compound.

14. The method of claim 13, wherein the medical device is a stent.

15. The method of claim 14, wherein the stent is implanted at a vascular treatment site at risk for restenosis.

16. The method of claim 6, wherein the composition coating the stent additionally comprises an agent that inhibits cell proliferation selected from the group consisting of Taxol™, Taxol™ analogs, Erbulozole, Dolastatin 10, Mivobulin isethionate, Vincristine, NSC-639829, Discodermolide, ABT-751, Altorhyrtins, Spongistatins, Cemadotin hydrochloride, Epothilone A, Epothilone B, Epothilone C, Epothilone D, Epothilone E, Epothilone F, Epothilone B N-oxide, Epothilone A N-oxide, 16-aza-epothilone B, 21-aminoepothilone B, 21-hydroxyepothilone D, 26-fluoroepothilone, Auristatin PE, Soblidotin, LS-4559-P, LS-4578, LS-4477, LS-4559, RPR-112378, Vincristine sulfate, DZ-3358, FR-182877, GS-164, GS-198, KAR-2, BSF-223651, SAH-49960, SDZ-268970, AM-97, AM-132, AM-138, IDN-5005, Cryptophycin 52, AC-7739, AC-7700, Vitilevuamide, Tubulysin A, Canadensol, Centaureidin, T-138067, COBRA-1, H10, H16, Oncocidin A1, DDE-313, Fijianolide B, Laulimalide, SPA-2, SPA-1, 3-IAABU, Narcosine, Nascapine, D-24851, A-105972, Hemiasterlin, 3-BAABU, TMPN, Vanadocene acetylacetonate, T-138026, Monsatrol, Inanocine, 3-IAABE, A-204197, T-607, RPR-115781, Desmethyleleutherobin, Desaetyleleutherobin, Isoeleutherobin A, Z-Eleutherobin, Caribaeoside, Caribaeolin, Halichondrin B, D-64131, D-68144, Diazonamide A, A-293620, NPI-2350, Taccalonolide A, TUB-245, A-259754, Diozostatin, (−)-Phenylahistin, D-68838, D-68836, Myoseverin B, D-4341 1, A-289099, A-318315, HTI-286, D-82317, D-82318, SC-12983, Resverastatin phosphate sodium, BPR-0Y-007, and SSR-250411.

17. The method of claim 6, wherein the compound is administered as a monotherapy.

18. The method of claim 6, wherein the compound is administered in combination with an agent that inhibits cell proliferation selected from the group consisting of Taxol™, Taxol™ analogs, Erbulozole, Dolastatin 10, Mivobulin isethionate, Vincristine, NSC-639829, Discodermolide, ABT-751, Altorhyrtins, Spongistatins, Cemadotin hydrochloride, Epothilone A, Epothilone B, Epothilone C, Epothilone D, Epothilone E, Epothilone F, Epothilone B N-oxide, Epothilone A N-oxide, 16-aza-epothilone B, 21-aminoepothilone B, 21-hydroxyepothilone D, 26-fluoroepothilone, Auristatin PE, Soblidotin, LS-4559-P, LS-4578, LS-4477, LS-4559, RPR-112378, Vincristine sulfate, DZ-3358, FR-182877, GS-164, GS-198, KAR-2, BSF-223651, SAH-49960, SDZ-268970, AM-97, AM-132, AM-138, IDN-5005, Cryptophycin 52, AC-7739, AC-7700, Vitilevuamide, Tubulysin A, Canadensol, Centaureidin, T-138067, COBRA-1, H10, H16, Oncocidin A1, DDE-313, Fijianolide B, Laulimalide, SPA-2, SPA-1, 3-IAABU, Narcosine, Nascapine, D-24851, A-105972, Hemiasterlin, 3-BAABU, TMPN, Vanadocene acetylacetonate, T-138026, Monsatrol, Inanocine, 3-IAABE, A-204197, T-607, RPR-115781, Desmethyleleutherobin, Desaetyleleutherobin, Isoeleutherobin A, Z-Eleutherobin, Caribaeoside, Caribaeolin, Halichondrin B, D-64131, D-68144, Diazonamide A, A-293620, NPI-2350, Taccalonolide A, TUB-245, A-259754, Diozostatin, (−)-Phenylahistin, D-68838, D-68836, Myoseverin B, D-43411, A-289099, A-318315, HTI-286, D-82317, D-82318, SC-12983, Resverastatin phosphate sodium, BPR-0Y-007, and SSR-250411.

19. The method of claim 6, wherein the compound is a disodium or dipotassium salt.

20. The method of claim 6 wherein Z is O, R₁ and R₂ are the same and R₃ and R₄ are the same.

21. The method of claim 20, wherein:
Y is a covalent bond, —C(R₅R₆)—, —(CH₂CH₂)—, trans-(CH═CH)—, cis-(CH═CH)— or —(CÿC)—; and
R₅ and R₆ are each independently —H, an aliphatic or substituted aliphatic group, or R₅ is —H and R₆ is an optionally substituted aryl group, or, R₅ and R₆, taken together, are an optionally substituted C2-C6 alkylene group.

22. The method of claim 21, wherein:
Y is —C(R₅R₆)—;
R₁ and R₂ are each an optionally substituted aryl group; and
R₃ and R₄ are each an optionally substituted aliphatic group.

23. The method of claim 22, wherein R₅ is —H and R₆ is —H, an aliphatic or substituted aliphatic group.

24. The method of claim 23, wherein R₃ and R₄ are each an alkyl group and R₆ is —H or methyl.

25. The method of claim 24, wherein R₁ and R₂ are each an optionally substituted phenyl group and R₃ and R₄ are each methyl or ethyl.

26. The method of claim 25, wherein the phenyl group represented by R₁ and the phenyl group represented by R₂ are optionally substituted with one or more groups selected from: —R^{a}, —OH, —Br, —Cl, —I, —F, —OR^{a}, —O—COR^{a}, —COR^{a}, —CN, —NCS, —NO₂, —COOH, —SO₃H, —NH₂, —NHR^{a}, —N(R^{a}R^{b}), —COOR^{a}, —CHO, —CONH₂, —CONHR^{a}, —CON(R^{a}R^{b}), —NHCOR^{a}, —NR^{c}COR^{a}, —NHCONH₂, —NHCONR^{a}H, —NHCON(R^{a}R^{b}), —NR^{c}CONH₂, —NR^{c}CONR^{a}H, —NR^{c}CON(R^{a}R^{b}), —C(═NH)—NH₂, —C(═NH)—NHR^{a}, —C(═NH)—N(R^{a}R^{b}), —C(═NR^{c})—NH₂, —C(═NR^{c})—NHR^{a}, —C(═NR^{c})—N(R^{a}R^{b}), —NH—C(═NH)—NH₂, —NH—C(═NH)—NHR^{a}, —NH—C(═NH)—N(R^{a}R^{b}), —NH—C(═NR^{c})—NH₂, —NH—C(═NR^{c})—NHR^{a}, —NH—C(═NR^{c})—N(R^{a}R^{b}), —NR^{d}H—C(═NH)—NH₂, —NR^{d}—C(═NH)—NHR^{a}, —NR^{d}—C(═NH)—N(R^{a}R^{b}), —NR^{d}—C(═NR^{c})—NH₂, —NR^{d}—C(═NR^{c})—NHR^{a}, —NR^{d}—C(═NR^{c})—N(R^{a}R^{b}), —NHNH₂, —NHNHR^{a}, —NHR^{a}R^{b}, —SO₂NH₂, —SO₂NHR^{a}, —SO₂NR^{a}R^{b}, —CH═CHR^{a}, —CH═CR^{a}R^{b}, —CRC═CR^{a}R^{b},—CR^{c}═CHR^{a}, —CR^{c}═CR^{a}R^{b}, —CCR^{a}, —SH, —SR^{a}, —S(O)R^{a}, —S(O)₂R^{a}, wherein R^{a}-R^{d} are each independently an alkyl group, aromatic group, non-aromatic heterocyclic group; or, —N(R^{a}R^{b}), taken together, form an optionally substituted non-aromatic heterocyclic group, wherein the alkyl, aromatic and non-aromatic heterocyclic group represented by R^{a}-R^{d} and the non-aromatic heterocyclic group represented by —N(R^{a}R^{b}) are each optionally and independently substituted with one or more groups represented by R^{#}, wherein R^{#} is R⁺, —OR⁺, —O(haloalkyl), —SR⁺, —NO₂, —CN, —NCS, —N(R⁺)₂, —NHCO₂R⁺, —NHC(O)R⁺, —NHNHC(O)R⁺, —NHC(O)N(R⁺)₂, —NHNHC(O)N(R⁺)₂, —NHNHCO₂R⁺, —C(O)C(O)R⁺, —C(O)CH₂C(O)R⁺, —CO₂R⁺, —C(O)R⁺, C(O)N(R⁺)₂, —OC(O)R⁺, —OC(O)N(R⁺)₂, —S(O)₂R⁺, —SO₂N(R⁺)₂, —S(O)R⁺, —NHSO₂N(R⁺)₂, —NHSO₂R⁺, —C(═S)N(R⁺)₂, or —C(═NH)—N(R⁺)₂; wherein R⁺ is —H, a C1-C4 alkyl group, a monocyclic heteroaryl group, a non-aromatic heterocyclic group or a phenyl group optionally substituted with alkyl, haloalkyl, alkoxy, haloalkoxy, halo, —CN, —NO₂, amine, alkylamine or dialkylamine; or —N(R⁺)₂ is a non-aromatic heterocyclic group, provided that non-aromatic heterocyclic groups represented by R⁺ and —N(R⁺)₂ that comprise a secondary ring amine are optionally acylated or alkylated.

27. The method of claim 26, wherein the phenyl groups represented by R₁ and R₂ are optionally substituted with C1-C4 alkyl, C1-C4 alkoxy, C1-C4 haloalkyl, C1-C4 haloalkoxy, phenyl, benzyl, pyridyl, —OH, —NH₂, —F, —Cl, —Br, —I, —NO₂ or —CN.

28. The method of claim 21, wherein:
Y is —CR₅R₆—;
R₁ and R₂ are both an optionally substituted aliphatic group;
R₅ is —H; and
R₆ is —H or an optionally substituted aliphatic group.

29. The method of claim 28, wherein R₁ and R₂ are both a C3-C8 cycloalkyl group optionally substituted with at least one alkyl group.

30. The method of claim 29, wherein R₃ and R₄ are both an alkyl group; and R₆ is —H or methyl.

31. The method of claim 30, wherein R₁ and R₂ are both cyclopropyl or 1-methylcyclopropyl.

32. A method of treating a proliferative vascular disorder in a subject, comprising administering to the subject an effective amount of a compound represented by the following Structural Formula: 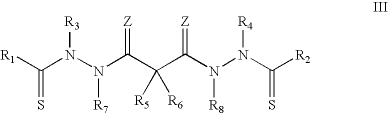
or a pharmaceutically acceptable salt or solvate thereof, wherein:
R₇-R₈ are both —H, and:
R₁ and R₂ are both phenyl, R₃ and R₄ are both methyl, and R₅ and R₆ are both —H;
R₁ and R₂ are both phenyl, R₃ and R₄ are both ethyl, and R₅ and R₆ are both —H;
R₁ and R₂ are both 4-cyanophenyl, R₃ and R₄ are both methyl, R₅ is methyl, and R₆ is —H;
R₁ and R₂ are both 4-methoxyphenyl, R₃ and R₄ are both methyl, and R₅ and R₆ are both —H;
R₁ and R₂ are both phenyl, R₃ and R₄ are both methyl, R₅ is methyl, and R₆ is —H;
R₁ and R₂ are both phenyl, R₃ and R₄ are both ethyl, R₅ is methyl, and R₆ is —H;
R₁ and R₂ are both 4-cyanophenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both ₃-cyanophenyl, R₃ and R₄ are both methyl, and R₅ and R₆ are both —H;
R₁ and R₂ are both ₃-fluorophenyl, R₃ and R₄ are both methyl, and R₅ and R₆ are both —H;
R₁ and R₂ are both 4-chlorophenyl, R₃ and R₄ are both methyl, R₅ is methyl, and R₆ is —H;
R₁ and R₂ are both 2-dimethoxyphenyl, R₃ and R₄ are both methyl, and R₅ and R₆ are both —H;
R₁ and R₂ are both 3-methoxyphenyl, R₃ and R₄ are both methyl, and R₅ and R₆ are both —H;
R₁ and R₂ are both 2,3-dimethoxyphenyl, R₃ and R₄ are both methyl, and R₅ and R₆ are both —H;
R₁ and R₂ are both 2,3-dimethoxyphenyl, R₃ and R₄ are both methyl, R₅ is methyl, and R₆ is —H;
R₁ and R₂ are both 2,5-difluorophenyl, R₃ and R₄ are both methyl, and R₅ and R₆ are both —H;
R₁ and R₂ are both 2,5-difluorophenyl, R₃ and R₄ are both methyl, R₅ is methyl, and R₆ is —H;
R₁ and R₂ are both 2,5-dichlorophenyl, R₃ and R₄ are both methyl, and R₅ and R₆ are both —H;
R₁ and R₂ are both 2,5-dimethylphenyl, R₃ and R₄ are both methyl, and R₅ and R₆ are both —H;
R₁ and R₂ are both 2,5-dimethoxyphenyl, R₃ and R₄ are both methyl, and R₅ and R₆ are both —H;
R₁ and R₂ are both phenyl, R₃ and R₄ are both methyl, and R₅ and R₆ are both —H;
R₁ and R₂ are both 2,5-dimethoxyphenyl, R₃ and R₄ are both methyl, R₅ is methyl, and R₆ is —H;
R₁ and R₂ are both cyclopropyl, R₃ and R₄ are both methyl, and R₅ and R₆ are both —H;
R₁ and R₂ are both cyclopropyl, R₃ and R₄ are both ethyl, and R₅ and R₆ are both —H;
R₁ and R₂ are both cyclopropyl, R₃ and R₄ are both methyl, R₅ is methyl, and R₆ is —H;
R₁ and R₂ are both 1-methylcyclopropyl, R₃ and R₄ are both methyl, and R₅ and R₆ are both —H;
R₁ and R₂ are both 1-methylcyclopropyl, R₃ and R₄ are both methyl, R₅ is methyl and R₆ is —H;
R₁ and R₂ are both 1-methylcyclopropyl, R₃ and R₄ are both methyl, R₅ is ethyl, and R₆ is —H;
R₁ and R₂ are both 1-methylcyclopropyl, R₃ and R₄ are both methyl, R₅ is n-propyl, and R₆ is —H;
R₁ and R₂ are both 1-methylcyclopropyl, R₃ and R₄ are both methyl, and R₅ and R₆ are both methyl;
R₁ and R₂ are both 1-methylcyclopropyl, R₃ and R₄ are both ethyl, and R₅ and R₆ are both —H;
R₁ and R₂ are both 1-methylcyclopropyl, R₃ is methyl, R₄ is ethyl, and R₅ and R₆ are both —H;
R₁ and R₂ are both 2-methylcyclopropyl, R₃ and R₄ are both methyl, and R₅ and R₆ are both —H;
R₁ and R₂ are both 2-phenylcyclopropyl, R₃ and R₄ are both methyl, and R₅ and R₆ are both —H;
R₁ and R₂ are both 1-phenylcyclopropyl, R₃ and R₄ are both methyl, and R₅ and R₆ are both —H;
R₁ and R₂ are both cyclobutyl, R₃ and R₄ are both methyl, and R₅ and R₆ are both —H;
R₁ and R₂ are both cyclopentyl, R₃ and R₄ are both methyl, and R₅ and R₆ are both —H;
R₁ and R₂ are both cyclohexyl, R₃ and R₄ are both methyl, and R₅ and R₆ are both —H;
R₁ and R₂ are both cyclohexyl, R₃ and R₄ are both phenyl, and R₅ and R₆ are both —H;
R₁ and R₂ are both methyl, R₃ and R₄ are both methyl, and R₅ and R₆ are both —H;
R₁ and R₂ are both methyl, R₃ and R₄ are both t-butyl, and R₅ and R₆ are both —H;
R₁ and R₂ are both methyl, R₃ and R₄ are both phenyl, and R₅ and R₆ are both —H;
R₁ and R₂ are both t-butyl, R₃ and R₄ are both methyl, and R₅ and R₆ are both —H;
R₁ and R₂ are ethyl, R₃ and R₄ are both methyl, and R₅ and R₆ are both —H; or
R₁ and R₂ are both n-propyl, R₃ and R₄ are both methyl, and R₅ and R₆ are both —H.

33. The method of claim 31, wherein the compound is a disodium salt.

34. The method of claim 32, wherein the proliferative vascular disorder is treated by implanting a stent at a vascular treatment site, wherein the stent comprises a reservoir, a coating composition, or a controlled release polymer matrix that comprises the compound and releases the compound in vivo.

35. A method of treating a proliferative vascular disorder in a subject, comprising administering to the subject an effective amount of a compound selected from: 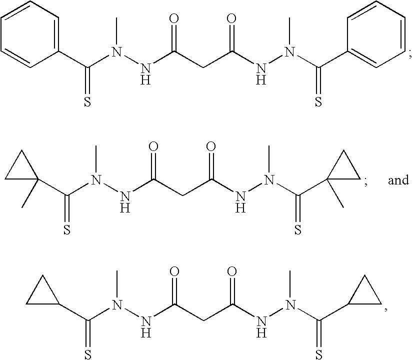
or a pharmaceutically acceptable salt or solvate thereof.

36. The method of claim 35, wherein the compound is a disodium salt.

37. The method of claim 35, wherein the compound is administered to a vascular treatment site in the subject.

38. The method of claim 37, wherein the compound is administered to the vascular treatment site by implanting a stent at the site, wherein the stent has a reservoir, a coating composition or a controlled release polymer matrix that comprises the compound and releases the compound in vivo.

39. The method of claim 38, wherein the stent is coated with a composition that comprises the compound and releases the compound in vivo.

40. The method of claim 38, wherein the reservoir, the coating composition or the controlled release polymer matrix additionally comprises and releases in vivo an agent that inhibits cell proliferation, wherein the agent is selected from the group consisting of Taxol™, Taxol™ analogs, Erbulozole, Dolastatin 10, Mivobulin isethionate, Vincristine, NSC-639829, Discodermolide, ABT-751, Altorhyrtins, Spongistatins, Cemadotin hydrochloride, Epothilone A, Epothilone B, Epothilone C, Epothilone D, Epothilone E, Epothilone F, Epothilone B N-oxide, Epothilone A N-oxide, 16-aza-epothilone B, 21-aminoepothilone B, 21-hydroxyepothilone D, 26-fluoroepothilone, Auristatin PE, Soblidotin, LS-4559-P, LS-4578, LS-4477, LS-4559, RPR-112378, Vincristine sulfate, DZ-3358, FR-182877, GS-164, GS-198, KAR-2, BSF-223651, SAH-49960, SDZ-268970, AM-97, AM-132, AM-138, IDN-5005, Cryptophycin 52, AC-7739, AC-7700, Vitilevuamide, Tubulysin A, Canadensol, Centaureidin, T-138067, COBRA-1, H10, H16, Oncocidin A1, DDE-313, Fijianolide B, Laulimalide, SPA-2, SPA-1, 3-IAABU, Narcosine, Nascapine, D-24851, A-105972, Hemiasterlin, 3-BAABU, TMPN, Vanadocene acetylacetonate, T-138026, Monsatrol, Inanocine, 3-IAABE, A-204197, T-607, RPR-115781, Desmethyleleutherobin, Desaetyleleutherobin, Isoeleutherobin A, Z-Eleutherobin, Caribaeoside, Caribaeolin, Halichondrin B, D-64131, D-68144, Diazonamide A, A-293620, NPI-2350, Taccalonolide A, TUB-245, A-259754, Diozostatin, (−)-Phenylahistin, D-68838, D-68836, Myoseverin B, D-43411, A-289099, A-318315, HTI-286, D-82317, D-82318, SC-12983, Resverastatin phosphate sodium, BPR-0Y-007, and SSR-250411.

41. A medical device comprising a reservoir, a coating composition or a controlled release polymer matrix that comprises a compound represented by the following Structural Formula: 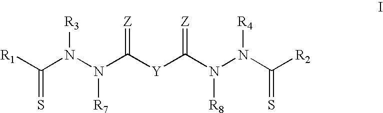
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁ -R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S,
wherein the compound is released in vivo.

42.-63. (canceled)

64. A method of treating a proliferative cell disorder at a treatment site in a subject, comprising contacting the subject with a medical device that comprises a reservoir, a coating composition or a controlled release polymer matrix comprises a compound represented by the following Structural Formula: 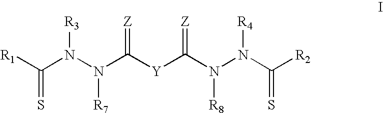
or a pharmaceutically acceptable salt or solvate thereof, wherein:
Y is a covalent bond or an optionally substituted straight chained hydrocarbyl group, or, Y, taken together with both >C=Z groups to which it is bonded, is an optionally substituted aromatic group;
R₁-R₄ are independently —H, an optionally substituted aliphatic group, an optionally substituted aryl group, or R₁ and R₃ taken together with the carbon and nitrogen atoms to which they are bonded, and/or R₂ and R₄ taken together with the carbon and nitrogen atoms to which they are bonded, form a non-aromatic heterocyclic ring optionally fused to an aromatic ring;
R₇-R₈ are independently —H, an optionally substituted aliphatic group, or an optionally substituted aryl group; and
Z is O or S; and
releasing the compound in vivo.

65.-87. (canceled)
